(19) United States
(12) Patent Application Publication
(10) Pub. No.: US 2024/0025998 A1
(43) Pub. Date: Jan. 25, 2024

(54) Methods of Treating Cancer with Anti-TIGIT Antibodies

(71) Applicant: Seagen Inc., Bothell, WA (US)

(72) Inventors: Shyra Gardai, Monroe, WA (US); Alyson Smith, Monroe, WA (US)

(73) Assignee: Seagen Inc., Bothell, WA (US)

(21) Appl. No.: 18/478,542

(22) Filed: Sep. 29, 2023

Related U.S. Application Data
(63) Continuation of application No. PCT/US22/23973, filed on Apr. 8, 2022.
(60) Provisional application No. 63/173,216, filed on Apr. 9, 2021.

(51) Int. Cl. C07K 16/28; A61P 35/00

(52) U.S. Cl. CPC C07K 16/2803; C07K 16/2827; C07K 16/2818; A61P 35/00; C07K 2317/72; C07K 2317/565

(57) ABSTRACT

Provided herein are methods of treating cancer with an anti-TIGIT antibody in combination with an anti-PD-1 antibody and/or an anti-PD-L1 antibody.

### CROSS-REFERENCE TO RELATED APPLICATIONS

This application is a continuation of International Application No. PCT/US2022/023973, filed Apr. 8, 2022, which claims the benefit of priority of U.S. Provisional Application No. 63/173,216, filed Apr. 9, 2021, each of which is incorporated by reference herein in its entirety for any purpose.

### SEQUENCE LISTING

This application contains a Sequence Listing, which has been submitted electronically in XML format. Said XML copy, created on Sep. 29, 2023, is named “2023-09-29_01218-0028-00PCT_ST26.xml” and is 25,868 bytes in size. The information in the Sequence Listing is incorporated herein by reference in its entirety.

### FIELD

Provided herein are methods of treating cancer with an anti-TIGIT antibody in combination with an anti-PD-1 antibody and/or an anti-PD-L1 antibody.

### BACKGROUND

TIGIT (“T-cell immunoreceptor with Ig and ITIM domains”) is an immune cell engager that is expressed on subsets of T cells, such as activated, memory, and regulatory T cells and natural killer (NK) cells. TIGIT is a member of the CD28 family within the Ig superfamily of proteins, and serves as a co-inhibitory molecule that limits T cell proliferation and activation and NK cell function. TIGIT mediates its immunosuppressive effect by competing with CD226 (also known as DNAX Accessory Molecule-1, or “DNAM-1”) for the same set of ligands: CD155 (also known as poliovirus receptor or “PVR”) and CD112 (also known as poliovirus receptor-related 2 or “PVRL2”). Levin et al., Eur. Immunol., 2011, 41:902-915. Because the affinity of CD155 for TIGIT is higher than its affinity for CD226, in the presence of TIGIT CD226 signaling is inhibited, thereby limiting T cell proliferation and activation.

In patients with certain cancers, such as melanoma, TIGIT expression is upregulated on tumor antigen (TA)-specific CD8+ T cells and CD8+ tumor-infiltrating lymphocytes (TILs). Blockade of TIGIT in the presence of TIGIT ligand (CD155)-expressing cells increases the proliferation, cytokine production, and degranulation of both TA-specific CD8+ T cells and CD8+ TILs. Chauvin et al., J Clin Invest., 2015, 125:2046-2058. Thus, TIGIT represents a potential therapeutic target for stimulating anti-tumor T cell responses in patients, although there remains a need for improved methods of blocking TIGIT and promoting anti-tumor responses, and a need for improved methods of treating cancer with anti-TIGIT antibodies, whether as a monotherapy or in combination with other agents (e.g., antibodies).

Improved methods of treating cancer with an anti-TIGIT antibody in combination with an anti-PD-1 antibody and/or an anti-PD-L1 antibody are provided.

### BRIEF SUMMARY

Embodiment 1. A method of treating cancer, comprising administering to a subject with cancer (1) an anti-TIGIT antibody, and (2) an anti-PD-1 antibody or an anti-PD-L1 antibody; wherein the level of PD-L1 in a sample of the cancer is less than 10 as measured by Combined Positive Score (CPS), or less than 50% as measured by Total Proportion Score (TPS), or less than 50% as measured by a Tumor Cell score (TC), or less than 10% as measured by Tumor-Infiltrating Immune Cell staining (IC), and wherein the anti-TIGIT antibody comprises an Fc region with enhanced effector function.

Embodiment 2. The method of embodiment 1, wherein the cancer expresses a level of PD-L1 that is less than 5, or less than 3, or less than 1, as measured by CPS.

Embodiment 3. The method of embodiment 1 or embodiment 2, wherein the cancer expresses a level of PD-L1 that is less than 40%, or less than 30%, or less than 20%, or less than 10%, or less than 5%, or less than 3%, or less than 1%, as measured by TPS.

Embodiment 4. The method of any one of embodiments 1-3, wherein the cancer expresses a level of PD-L1 that is less than 40%, or less than 30%, or less than 20%, or less than 10%, or less than 5%, or less than 3%, or less than 1%, as measured by TC.

Embodiment 5. The method of any one of embodiments 1-4, wherein the cancer expresses a level of PD-L1 that is less than 5%, or less than 3%, or less than 1%, as measured by IC.

Embodiment 6. The method of any one of embodiments 1-5, wherein:
a) the cancer is non-small lung cancer, and the TPS is <1%;
b) the cancer is head and neck squamous cell cancer (HNSCC) and the CPS is <1;
c) the cancer is urothelial carcinoma and the CPS is <10;
d) the cancer is gastric cancer and the CPS is <1;
e) the cancer is esophageal cancer and the CPS<10;
f) the cancer is cervical cancer and the CPS<1; or
g) the cancer is triple negative breast cancer, and the CPS<10.

Embodiment 7. The method of embodiment 6, wherein the method comprises administering an anti-PD-1 antibody, wherein the anti-PD-1 antibody is pembrolizumab or nivolumab.

Embodiment 8. The method of any one of embodiments 1-5, wherein the cancer is non-small cell lung cancer, and the TPS is <50%.

Embodiment 9. The method of embodiment 8, the method comprises administering an anti-PD-1 antibody, wherein the anti-PD-1 antibody is cemiplimab.

Embodiment 10. The method of any one of embodiments 1-5, wherein:
a) the cancer is urothelial carcinoma and IC is <5%;
b) the cancer is triple-negative breast cancer and IC is <1%; or
c) the cancer is non-small cell lung cancer and IC is <10%; or
d) the cancer is non-small cell lung cancer and TC<50%.

Embodiment 11. The method of embodiment 10, the method comprises administering an anti-PD-1 antibody, wherein the anti-PD-1 antibody is atezolizumab.

Embodiment 12. The method of any one of embodiments 1-11, wherein the anti-PD-1 antibody or anti-PD-L1 antibody is administered at a sub-therapeutic dose.

Embodiment 13. A method of treating cancer, comprising administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the anti-PD-1 antibody or anti-PD-L1 antibody is administered at a sub-therapeutic dose.

Embodiment 14. The method of embodiment 12 or embodiment 13, wherein the sub-therapeutic dose of the anti-PD-1 antibody or anti-PD-L1 antibody: a) is lower than the monotherapy dose of the antibody for the cancer being treated and/or b) comprises less frequent dosing of the antibody than the frequency of monotherapy dosing for the cancer being treated.

Embodiment 15. The method of any one of embodiments 12-14, wherein the sub-therapeutic dose of the antibody includes a dose that is lower than the monotherapy dose of the antibody for the cancer being treated.

Embodiment 16. The method of embodiment 15, wherein the sub-therapeutic dose is a dose of the antibody that is between 5% and 90%, or 5% and 80%, or 5% and 70%, or 5% and 60%, or 5% and 50%, or 5% and 40%, or 5% and 30% of the monotherapy dose for the cancer being treated.

Embodiment 17. The method of any one of embodiments 14-16, wherein the method comprises administering an anti-PD-1 antibody, wherein the anti-PD-1 antibody is pembrolizumab, and wherein the monotherapy dose is 200 mg or 400 mg.

Embodiment 18. The method of any one of embodiments 14-16, wherein the method comprises administering an anti-PD-1 antibody, wherein the anti-PD-1 antibody is nivolumab, and wherein the monotherapy dose is 240 mg, 360 mg, or 480 mg.

Embodiment 19. The method of any one of embodiments 14-16, wherein the method comprises administering an anti-PD-1 antibody, wherein the anti-PD-1 antibody is cemiplimab, and wherein the monotherapy dose is 350 mg.

Embodiment 20. The method of any one of embodiments 14-16, wherein the method comprises administering an anti-PD-L1 antibody, wherein the anti-PD-L1 antibody is avelumab, and wherein the monotherapy dose is 800 mg.

Embodiment 21. The method of any one of embodiments 14-16, wherein the method comprises administering an anti-PD-L1 antibody, wherein the anti-PD-L1 antibody is durvalumab, and wherein the monotherapy dose is 10 mg/kg or 1500 mg.

Embodiment 22. The method of any one of embodiments 14-16, wherein the method comprises administering an anti-PD-L1 antibody, wherein the anti-PD-L1 antibody is atezolizumab, and wherein the monotherapy dose is 840 mg, 1200 mg, or 1680 mg.

Embodiment 23. The method of any one of embodiments 12-22, wherein the sub-therapeutic dose of the antibody comprises less frequent dosing of the antibody than the frequency of monotherapy dosing for the cancer being treated.

Embodiment 24. The method of embodiment 23, wherein the method comprises administering an anti-PD-1 antibody, wherein the anti-PD-1 antibody is pembrolizumab, and wherein the frequency of monotherapy dosing is every 3 weeks or every 6 weeks.

Embodiment 25. The method of embodiment 24, wherein the method comprises administering an anti-PD-1 antibody, wherein the anti-PD-1 antibody is pembrolizumab, and wherein the monotherapy dose is 200 mg every 3 weeks or 400 mg every 6 weeks.

Embodiment 26. The method of embodiment 23, wherein the method comprises administering an anti-PD-1 antibody, wherein the anti-PD-1 antibody is nivolumab, and wherein the frequency of monotherapy dosing is every 2 weeks or every 3 weeks or every 4 weeks.

Embodiment 27. The method of embodiment 26, wherein the method comprises administering an anti-PD-1 antibody, wherein the anti-PD-1 antibody is nivolumab, and wherein the monotherapy dose is 240 mg every 2 weeks, 360 mg every 3 weeks, or 480 mg every 4 weeks.

Embodiment 28. The method of embodiment 23, wherein the method comprises administering an anti-PD-1 antibody, wherein the anti-PD-1 antibody is cemiplimab, and wherein the frequency of monotherapy dosing is every 3 weeks.

Embodiment 29. The method of embodiment 23, wherein the method comprises administering an anti-PD-L1 antibody, wherein the anti-PD-L1 antibody is avelumab, wherein the frequency of monotherapy dosing is every 2 weeks.

Embodiment 30. The method of embodiment 23, wherein the method comprises administering an anti-PD-L1 antibody, wherein the anti-PD-L1 antibody is durvalumab, wherein the frequency of monotherapy dosing is every 2 weeks or every 4 weeks.

Embodiment 31. The method of embodiment 30, wherein the method comprises administering an anti-PD-L1 antibody, wherein the anti-PD-L1 antibody is durvalumab, and wherein the monotherapy dose is 10 mg/kg mg every 2 weeks or 1500 mg every 4 weeks.

Embodiment 32. The method of embodiment 23, wherein the method comprises administering an anti-PD-L1 antibody, wherein the anti-PD-L1 antibody is atezolizumab, wherein the frequency of monotherapy dosing is every 2 weeks, every 3 weeks, or every 4 weeks.

Embodiment 33. The method of embodiment 32, wherein the method comprises administering an anti-PD-L1 antibody, wherein the anti-PD-L1 antibody is atezolizumab, and wherein the monotherapy dose is 840 mg every 2 weeks, 1200 mg every 3 weeks, or 1680 mg every 4 weeks.

Embodiment 34. The method of any one of embodiments 1-33, wherein the cancer is selected from small cell lung cancer, early-stage small cell lung cancer, renal cell carcinoma, urothelial cancer, triple negative breast cancer, gastric cancer, hepatocellular carcinoma, glioblastoma, ovarian cancer, head and neck squamous cell carcinoma, esophageal squamous cell carcinoma (ESCC), and non-microsatellite instability high (non-MSI high) colorectal cancer.

Embodiment 35. A method of treating cancer, comprising administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer is selected from small cell lung cancer, early-stage small cell lung cancer, renal cell carcinoma, urothelial cancer, triple negative breast cancer, gastric cancer, hepatocellular carcinoma, glioblastoma, ovarian cancer, head and neck squamous cell carcinoma, esophageal squamous cell carcinoma (ESCC), and non-microsatellite instability high (non-MSI high) colorectal cancer.

Embodiment 36. The method of embodiment 34 or embodiment 35, wherein the method is first line treatment of urothelial cancer.

Embodiment 37. The method of any one of embodiments 1-36, wherein the cancer comprises a mutation that reduces the efficacy of the anti-PD-1 antibody or anti-PD-L1 antibody.

Embodiment 38. A method of treating cancer, comprising administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer comprises a mutation that reduces the efficacy of the anti-PD-1 antibody or anti-PD-L1 antibody.

Embodiment 39. The method of embodiment 37 or embodiment 38, wherein the cancer comprises a mutation in an EGFR gene and/or a mutation in an ALK gene and/or a mutation in the ROS1 gene.

Embodiment 40. The method of any one of embodiments 37-39, wherein the cancer is non-small cell lung cancer, and wherein the cancer comprises a mutation in an EGFR gene and/or a mutation in an ALK gene.

Embodiment 41. The method of embodiment 40, wherein the method comprises administering an anti-PD-1 antibody, wherein the anti-PD-1 antibody is pembrolizumab or nivolumab; or wherein the method comprises administering an anti-PD-L1 antibody, wherein the anti-PD-L1 antibody is atezolizumab.

Embodiment 42. The method of any one of the preceding embodiments, wherein the anti-TIGIT antibody comprises an Fc with enhanced binding to at least one of FcγRIIIa, FcγRIIa, and FcγRI.

Embodiment 43. The method of embodiment 42, wherein the anti-TIGIT antibody comprises an Fc with enhanced binding to at least FcγRIIIa.

Embodiment 44. The method of embodiment 42, wherein anti-TIGIT antibody comprises an Fc with enhanced binding to at least FcγRIIIa and FcγRIIa.

Embodiment 45. The method of embodiment 42, wherein the anti-TIGIT antibody comprises an Fc with enhanced binding to at least FcγRIIIa and FcγRI.

Embodiment 46. The method of embodiment 42, wherein the anti-TIGIT antibody comprises an Fc with enhanced binding to FcγRIIIa, FcγRIIa, and FcγRI.

Embodiment 47. The method of any one of embodiments 42-46, wherein the Fc of the anti-TIGIT antibody has reduced binding to FcγRIIb.

Embodiment 48. The method of any one of the preceding embodiments, wherein the anti-TIGIT antibody comprises substitutions S293D, A330L, and I332E in the heavy chain constant region.

Embodiment 49. The method of any one of the preceding embodiments, wherein the anti-TIGIT antibody is nonfucosylated.

Embodiment 50. The method of any one of the preceding embodiments, wherein the method comprises administering a composition of anti-TIGIT antibodies, wherein at least 90%, at least 91%, at least 92%, at least 93%, at least 94%, at least 95%, at least 96%, at least 97%, at least 98%, or at least 99% of the antibodies in the composition are nonfucosylated.

Embodiment 51. The method of any one of the preceding embodiments, wherein the Fc of the anti-TIGIT antibody comprises an Fc with enhanced ADCC and/or ADCP activity relative to a corresponding wild-type Fc of the same isotype.

Embodiment 52. The method of any one of the preceding embodiments, wherein the anti-TIGIT antibody comprises:
a) a heavy chain CDR1 comprising an amino acid sequence selected from SEQ ID NOs: 7-9;
b) a heavy chain CDR2 comprising an amino acid sequence selected from SEQ ID NOs: 10-13;
c) a heavy chain CDR3 comprising an amino acid sequence selected from SEQ ID NOs: 14-16;
d) a light chain CDR1 comprising the amino acid sequence of SEQ ID NO: 17;
e) a light chain CDR2 comprising the amino acid sequence of SEQ ID NO: 18; and
f) a light chain CDR3 comprising the amino acid sequence of SEQ ID NO: 19.

Embodiment 53. The method of any one of the preceding embodiments, wherein the anti-TIGIT antibody comprises a heavy chain CDR1, CDR2, and CDR3 and a light chain CDR1, CDR, and CDR3 comprising the sequences of:
a) SEQ ID NOs: 7, 10, 14, 17, 18, and 19, respectively; or
b) SEQ ID NOs: 8, 11, 14, 17, 18, and 19, respectively; or
c) SEQ ID NOs: 9, 12, 15, 17, 18, and 19, respectively; or
d) SEQ ID NOs: 8, 13, 16, 17, 18, and 19, respectively; or
e) SEQ ID NOs: 8, 12, 16, 17, 18, and 19, respectively.

Embodiment 54. The method of any one of the preceding embodiments, wherein the anti-TIGIT antibody comprises a heavy chain variable region comprising an amino acid sequence selected from SEQ ID NOs: 1-5 and a light chain variable region comprising the amino acid sequence of SEQ ID NO: 6.

Embodiment 55. The method of any one of the preceding embodiments, wherein the anti-TIGIT antibody comprises a heavy chain comprising an amino acid sequence selected from SEQ ID NOs: 20-24 and a light chain comprising the amino acid sequence of SEQ ID NO: 25.

Embodiment 56. The method of any one of the preceding embodiments, wherein the anti-TIGIT antibody is administered at a sub-therapeutic dose.

Embodiment 57. The method of embodiment 56, wherein the sub-therapeutic dose of the anti-TIGIT antibody a) is lower than the monotherapy dose of the anti-TIGIT antibody for the cancer being treated and/or b) comprises less frequent dosing of the anti-TIGIT antibody than the frequency of monotherapy dosing for the cancer being treated.

Embodiment 58. The method of embodiment 56 or embodiment 57, wherein the sub-therapeutic dose of the anti-TIGIT antibody includes a dose that is lower than the monotherapy dose of the anti-TIGIT antibody for the cancer being treated.

Embodiment 59. The method of any one of embodiments 56-58, wherein the sub-therapeutic dose is a dose of the anti-TIGIT antibody that is between 5% and 90%, or 5% and 80%, or 5% and 70%, or 5% and 60%, or 5% and 50%, or 5% and 40%, or 5% and 30% of the monotherapy dose for the cancer being treated.

Embodiment 60. The method of any one of embodiments 56-59, wherein the sub-therapeutic dose of the anti-TIGIT antibody comprises less frequent dosing of the anti-TIGIT antibody than the frequency of monotherapy dosing for the cancer being treated.

Embodiment 61. The method of any one of the preceding embodiments, wherein the method comprises administering an anti-PD-1 antibody.

Embodiment 62. The method of embodiment 61, wherein the anti-PD-1 antibody is selected from pembrolizumab, nivolumab, CT-011, BGB-A317, cemiplimab, sintilimab, tislelizumab, TSR-042, PDR001, or toripalimab.

Embodiment 63. The method of any one of embodiments 1-60, wherein the method comprises administering an anti-PD-L1 antibody.

Embodiment 64. The method of embodiment 63, wherein the anti-PD-L1 antibody is selected from durvalumab, BMS-936559, atezolizumab, or avelumab.

### BRIEF DESCRIPTION OF THE DRAWINGS

FIGS. 1A-1C show the composition of immune cells in Renca tumors (FIG. 1A), CT26 tumors (FIG. 1B), and MC38 tumors (FIG. 1C) at 100 mm³ grown in fully immunocompetent mice.

FIGS. 2A-B show the mRNA expression levels of PD-1 (FIG. 2A) and PD-L1 (FIG. 2B) in these tumors.

FIG. 3 shows in vivo data for treatment with a sub-therapeutic dose of anti-TIGIT antibodies with Fc-backbones having distinct effector function in combination with a sub-therapeutic dose of an anti-PD-1 antibody against a subcutaneous syngeneic MC38 tumor.

FIGS. 4A and 4B show in vivo data for treatment with a sub-therapeutic dose of SEA-TGT mIgG2a antibody (i.e., the SEA-TGT antibody reformatted as a nonfucosylated mouse IgG2a that corresponds to a nonfucosylated human IgG1 backbone), which is a nonfucosylated effector function enhanced anti-TIGIT antibody, with a sub-therapeutic dose of an anti-PD-1 antibody, or with a combination of both, against a subcutaneous syngeneic CT26 tumor (FIG. 4A) or Renca tumor (FIG. 4B).

FIGS. 5A-5C show in vivo response data for single agent treatment with different anti-TIGIT antibodies at therapeutic doses against a subcutaneous syngeneic MC38 tumor (FIG. 5A), CT26 tumor (FIG. 5B), or Renca tumor (FIG. 5C). FIGS. 5D-5F show in vivo response data for single agent treatment with an anti-PD-1 antibody at therapeutic doses in the various syngeneic subcutaneous tumors MC38 (FIG. 5D), CT26 (FIG. 5E) or Renca (FIG. 5F).

### DETAILED DESCRIPTION

### I. Introduction

The present invention is based in part on the surprising finding that cancers expressing low levels of PD-L1 can be treated with an anti-TIGIT antibody in combination with an anti-PD-1 antibody and/or an anti-PD-L1 antibody. This was particularly found to be the case with anti-TIGIT antibodies having enhanced Fc binding characteristics and effector function. The desired Fc binding characteristics included activities such as enhanced binding to activating FcγRs, decreased binding to inhibitory FcγRs, enhanced ADCC activity, and/or enhanced ADCP activity. Certain such antibodies with the desired activities were nonfucosylated.

Based upon these findings, the inventors have demonstrated that administering an anti-TIGIT antibody in combination with an anti-PD-1 antibody and/or an anti-PD-L1 antibody to a subject whose cancer expresses a low level of PD-L1 results in reduction of tumor size and/or growth rate. In some embodiments, the antibodies may be administered at a sub-therapeutic dose. In various embodiments, the anti-TIGIT antibody has enhanced Fc binding characteristics and/or effector function.

Accordingly, some embodiments provided herein are methods of treating cancer which comprise administering to a subject with cancer (1) an anti-TIGIT antibody, and (2) an anti-PD-1 antibody or an anti-PD-L1 antibody; wherein the cancer expresses a level of PD-L1 that is less than 10 as measured by Combined Positive Score (CPS) or less than 50% as measured by Total Proportion Score (TPS), and wherein the anti-TIGIT antibody comprises an Fc region with enhanced effector function.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the anti-PD-1 antibody or anti-PD-L1 antibody is administered at a sub-therapeutic dose.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the anti-TIGIT antibody is administered at a sub-therapeutic dose.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein both the anti-TIGIT antibody and the anti-PD-1 or anti-PD-L1 antibody is administered at a sub-therapeutic dose.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer is selected from small cell lung cancer, early-stage small cell lung cancer, renal cell carcinoma, urothelial cancer, triple negative breast cancer, gastric cancer, hepatocellular carcinoma, glioblastoma, ovarian cancer, head and neck squamous cell carcinoma, esophageal squamous cell carcinoma (ESCC), and non-microsatellite instability high (non-MSI high) colorectal cancer.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer comprises a mutation that reduces the efficacy of the anti-PD-1 antibody or anti-PD-L1 antibody.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention.

As used herein, the singular forms “a”, “an” and “the” include plural referents unless the content clearly dictates otherwise. Thus, for example, reference to “an antibody” optionally includes a combination of two or more such molecules, and the like.

The term “about,” as used herein, refers to the usual error range for the respective value readily known to the skilled person in this technical field.

The term “antibody” includes intact antibodies and antigen-binding fragments thereof, wherein the antigen-binding fragments comprise the antigen-binding region and at least a portion of the heavy chain constant region comprising asparagine (N) 297, located in CH2. Typically, the “variable region” contains the antigen-binding region of the antibody and is involved in specificity and affinity of binding. See, FUNDAMENTAL IMMUNOLOGY 7^{TH} EDITION, Paul, ed., Wolters Kluwer Health/Lippincott Williams & Wilkins (2013). Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively.

The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “monoclonal antibody” refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, for example, U.S. Pat. No. 4,816,567). The “monoclonal antibodies” may also be isolated from phage antibody libraries using the techniques described in Clackson et al. (1991) Nature, 352:624-628 and Marks et al. (1991) J Mol. Biol., 222:581-597, for example or may be made by other methods. The antibodies described herein are monoclonal antibodies.

Specific binding of a monoclonal antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region, means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, FUNDAMENTAL IMMUNOLOGY (Paul, W., ed., 2nd ed. Raven Press, N.Y., 1989, Ch. 7, incorporated by reference in its entirety for all purposes).

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, SEQUENCES OF PROTEINS OF IMMUNOLOGICAL INTEREST (National Institutes of Health, Bethesda, M D, 1987 and 1991), or Chothia & Lesk, J. Mol. Biol. 196:901-917 (1987); Chothia et al., Nature 342:878-883 (1989), or a composite of Kabat and Chothia, or IMGT (ImMunoGeneTics information system), AbM or Contact or other conventional definition of CDRs. Kabat also provides a widely used numbering convention (Kabat numbering) in which corresponding residues between different heavy chains or between different light chains are assigned the same number. Unless otherwise apparent from the context, Kabat numbering is used to designate the position of amino acids in the variable regions. Unless otherwise apparent from the context, EU numbering is used to designated positions in constant regions.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

As used herein, the term “chimeric antibody” refers to an antibody molecule in which (a) the constant region, or a portion thereof, is replaced so that the antigen binding site (variable region, CDR, or portion thereof) is linked to a constant region of a different species.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., EPITOPE MAPPING PROTOCOLS, IN METHODS IN MOLECULAR BIOLOGY, VOL. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The phrase “specifically binds” refers to a molecule (e.g., antibody or antibody fragment) that binds to a target with greater affinity, avidity, more readily, and/or with greater duration to that target in a sample than it binds to a non-target compound. In some embodiments, an antibody that specifically binds a target is an antibody that binds to the target with at least 2-fold greater affinity than non-target compounds, such as, for example, at least 4-fold, 5-fold, 6-fold, 7-fold, 8-fold, 9-fold, 10-fold, 20-fold, 25-fold, 50-fold, or 100-fold greater affinity. For example, an antibody that specifically binds TIGIT will typically bind to TIGIT with at least a 2-fold greater affinity than to a non-TIGIT target. It will be understood by a person of ordinary skill in the art reading this definition, for example, that an antibody (or moiety or epitope) that specifically or preferentially binds to a first target may or may not specifically or preferentially bind to a second target. As such, “specific binding” does not necessarily require (although it can include) exclusive binding.

The term “binding affinity” is herein used as a measure of the strength of a non-covalent interaction between two molecules, e.g., an antibody, or fragment thereof, and an antigen. The term “binding affinity” is used to describe monovalent interactions (intrinsic activity).

Binding affinity between two molecules, e.g., an antibody, or fragment thereof, and an antigen, through a monovalent interaction may be quantified by determination of the dissociation constant (K_{D}). In turn, K_{D} can be determined by measurement of the kinetics of complex formation and dissociation using, as a nonlimiting example, the surface plasmon resonance (SPR) method (Biacore™). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those, for example, set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of ligands such as antibodies towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art or as described in the Examples section below, such as Surface Plasmon Resonance (SPR), e.g., by using a Biacore™ system; kinetic exclusion assays such as KinExA®; and BioLayer interferometry (e.g., using the ForteBio® Octet platform). In some embodiments, binding affinity is determined using a BioLayer interferometry assay. See, e.g., Wilson et al., Biochemistry and Molecular Biology Education, 38:400-407 (2010); Dysinger et al., J. Immunol. Methods, 379:30-41 (2012); and Estep et al., Mabs, 2013, 5:270-278.

The term “cross-reacts,” as used herein, refers to the ability of an antibody to bind to an antigen other than the antigen against which the antibody was raised. In some embodiments, cross-reactivity refers to the ability of an antibody to bind to an antigen from another species than the antigen against which the antibody was raised. As a non-limiting example, an anti-TIGIT antibody as described herein that is raised against a human TIGIT antigen can exhibit cross-reactivity with TIGIT from a different species (e.g., mouse or monkey).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle intended to facilitate its use. Interfering proteins and other contaminants can include, for example, cellular components of the cells from which an antibody is isolated or recombinantly produced. Sometimes monoclonal antibodies are at least 60%, 70%, 80%, 90%, 95 or 99% w/w pure of interfering proteins and contaminants from production or purification. The antibodies described herein, including rat, chimeric, veneered and humanized antibodies can be provided in isolated and/or purified form.

“Combined Positive Score” or “CPS” is an immunohistochemical method of measuring PD-L1 expression in a cancer, such as a tumor sample from a cancer. CPS is the number of PD-L1 staining cells (tumor cells, lymphocytes, macrophages) divided by the total number of viable tumor cells, multiplied by 100. For some therapeutic treatments, a tumor sample is considered to have PD-L1 expression if CPS≥1. For example, a CPS≥1 is required for a subject to be eligible for certain PD-1 or PD-L1 inhibitor therapies, such as subjects with gastric cancer, cervical cancer, and head and neck squamous cell cancer. In some instances, a CPS≥10 is required for a subject to be eligible for certain PD-1 or PD-L1 inhibitor therapies, such as subjects with urothelial cancer (bladder cancer), esophageal squamous cell carcinoma (ESCC), or triple-negative breast cancer being treated with pembrolizumab.

“Tumor Proportion Score” or “TPS” is an immunohistochemical method of measuring PD-L1 expression in a cancer, such as a tumor sample from a cancer. TPS is the percentage of viable tumor cells showing partial or complete membrane staining at any intensity. For some therapeutic treatments, a tumor sample is considered to have PD-L1 expression if TPS≥1% and high PD-L1 expression if TPS≥50%. For example, a TPS≥1% is the required for a subject to be eligible for certain PD-1 or PD-L1 inhibitor therapies (e.g., pembrolizumab), such as subjects with non-small cell lung cancer. In some instances, a TPS≥50% is the required for a subject to be eligible for certain PD-1 or PD-L1 inhibitor therapies (e.g., cemiplimab).

Tumor-Infiltrating Immune Cell (IC) staining or “IC” is an immunohistochemical method of measuring PD-L1 expression, such as a tumor sample from a cancer. The expression is measured as the proportion of tumor area that is occupied by PD-L1 staining IC of any intensity. If the specimen contains PD-L1 staining of any intensity in tumor infiltrating immune cells occupying ≥5% of tumor area, then the specimen is assigned a PD-L1 expression level of ≥5% IC. If the specimen contains PD-L1 staining of any intensity in tumor-infiltrating immune cells covering <5% of tumor area, then the specimen is assigned a PD-L1 expression level of <5% IC. For some therapeutic treatments, IC is used to score PD-L1 expression from urothelial carcinoma tissue. Urothelial carcinoma tissue samples obtained from resections, transurethral resection of bladder tumor (TURBT), and core needle biopsies from both primary and metastatic sites can be used in IC assays. Commercially available IC assays include the Ventana PD-L1 (SP142) Assay™.

A Tumor Cell or “TC” score refers to the percentage of PD-L1 expressing tumor cells (% TC) of any intensity, and is similar to TPS. In some embodiments, a TC score is obtained using the Ventana PD-L1 (SP142) Assay. TC scores are used, for example, when NSCLC patients are treated with atezolizumab (TECENTRIQ). In this indication, the threshold for treatment is a TC score of ≥50%. Further information on TC scoring is available, for example, in: 1) Physician Labeling: Ventana PD-L1 (SP142) Assay (2020) Ventana Medical Systems, Inc. and Roche Diagnostics International, Inc.; and 2) Ventana PD-L1 (SP142) Assay: Interpretation Guide (2019) Ventana Medical Systems, Inc. and Roche Diagnostics International, Inc.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision.

The terms “therapy,” “treatment,” and “amelioration” refer to any reduction in the severity of symptoms. In the case of treating cancer, treatment can refer to reducing, e.g., tumor size, number of cancer cells, growth rate, metastatic activity, cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent or severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

As used herein, a “therapeutic amount” or “therapeutically effective amount” of an agent (e.g., an antibody as described herein) is an amount of the agent that prevents, alleviates, abates, ameliorates, or reduces the severity of symptoms of a disease (e.g., a cancer) in a subject.

As used herein, a “sub-therapeutic amount” or “sub-therapeutic dose” of an agent (e.g., an antibody as described herein) is a dose of the agent that is less than the dose that is administered when the agent is used as a monotherapy to treat the same indication, such as the same type or subtype of cancer. A sub-therapeutic dose could include less frequent dosing of the monotherapy dose, such that the subject receives an overall lower dose of the agent.

The terms “administer,” “administered,” or “administering” refer to methods of delivering agents, compounds, or compositions to the desired site of biological action. These methods include, but are not limited to, topical delivery, parenteral delivery, intravenous delivery, intradermal delivery, intramuscular delivery, colonic delivery, rectal delivery, or intraperitoneal delivery. Administration techniques that are optionally employed with the agents and methods described herein, include e.g., as discussed in Goodman and Gilman, THE PHARMACOLOGICAL BASIS OF THERAPEUTICS, current ed.; Pergamon; and Remington's, PHARMACEUTICAL SCIENCES (current edition), Mack Publishing Co., Easton, PA.

### III. Expression Levels of PD-L1

The level of expression of PD-L1 in a cancer in a subject can be measured prior to administering any composition or utilizing any method disclosed herein. The level of expression can be determined by any methods known in the art.

In order to assess the level of expression of PD-L1, in some embodiments, a cancer tissue sample can be obtained from the subject who is in need of the therapy. In another embodiment, the assessment of level of expression of PD-L1 can be achieved without obtaining a cancer tissue sample. In some embodiments, selecting a suitable subject includes (i) optionally providing a cancer tissue sample obtained from a subject, the cancer tissue sample comprising cancer cells and/or cancer-infiltrating inflammatory cells; and (ii) assessing the proportion of cells in the cancer tissue sample that express PD-L1 on the surface of the cells.

In any of the methods comprising the measurement of PD-L1 expression in a cancer tissue sample, however, it should be understood that the step comprising the provision of a cancer tissue sample obtained from a subject is an optional step. It should also be understood that in certain embodiments the “measuring” or “assessing” step to identify, or determine the number or proportion of, cells in the cancer tissue sample that express PD-L1 on the cell surface is performed by a transformative method of assaying for PD-L1 expression, for example by performing a reverse transcriptase-polymerase chain reaction (RT-PCR) assay or an immunohistochemical (IHC) assay. In some embodiments, no transformative step is involved and PD-L1 expression is assessed by, for example, reviewing a report of test results from a laboratory. In certain embodiments, the steps of the methods up to, and including, assessing PD-L1 expression provides an intermediate result that may be provided to a physician or other healthcare provider for use in selecting a suitable subject for treatment. In certain embodiments, the steps that provide the intermediate result is performed by a medical practitioner or someone acting under the direction of a medical practitioner. In other embodiments, these steps are performed by an independent laboratory or by an independent person such as a laboratory technician.

In some embodiments, the proportion of cells that express PD-L1 is assessed by performing an assay to determine the presence of PD-L1 RNA. In some embodiments, the presence of PD-L1 RNA is determined by RT-PCR, in situ hybridization or RNase protection. In other embodiments, the proportion of cells that express PD-L1 is assessed by performing an assay to determine the presence of PD-L1 polypeptide. In some embodiments, the presence of PD-L1 polypeptide is determined by an IHC assay, an enzyme-linked immunosorbent assay (ELISA), in vivo imaging, or flow cytometry. In some embodiments, PD-L1 expression is determined by an IHC assay. See Chen et al., (2013) Clin. Cancer Res. 19(13): 3462-3473.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM) and multiphoton microscopy (MPM), may herald even greater use of these techniques in cancer research. Some of these molecular imaging systems allow clinicians to not only see where a cancer is located in the body, but also to visualize the expression and activity of specific molecules, cells, and biological processes that influence cancer behavior and/or responsiveness to therapeutic drugs (Condeelis and Weissleder, In vivo imaging in cancer, Cold Spring Harb. Perspect. Biol. 2(12): a003848 (2010)). Antibody specificity, coupled with the sensitivity and resolution of PET, makes immunoPET imaging particularly attractive for monitoring and assaying expression of antigens in tissue samples (McCabe and Wu, Positive progress in immunoPET—not just a coincidence, Cancer Biother. Radiopharm. 25(3):253-61 (2010); Olafsen et al., ImmunoPET imaging of B-cell lymphoma using 124I-anti-CD20 scFv dimers (diabodies), Protein Eng. Des. Sel. 23(4):243-9 (2010)). In certain embodiments, PD-L1 expression is assayed by immunoPET imaging. In certain embodiments, the proportion of cells in a cancer tissue sample that express PD-L1 is assessed by performing an assay to determine the presence of PD-L1 polypeptide on the surface of cells in the cancer tissue sample. In certain embodiments, the cancer tissue sample is a formalin-fixed paraffin-embedded (FFPE) tissue sample. In other embodiments, the presence of PD-L1 polypeptide is determined by an IHC assay. In further embodiments, the IHC assay is performed using an automated process. In some embodiments, the IHC assay is performed using an anti-PD-L1 monoclonal antibody to bind to the PD-L1 polypeptide.

In some embodiments, an automated IHC method is used to assay the expression of PD-L1 on the surface of cells in FFPE tissue specimens. This disclosure provides methods for detecting the presence of human PD-L1 antigen in a cancer tissue sample, or quantifying the level of human PD-L1 antigen or the proportion of cells in the sample that express the antigen, which methods comprise contacting the test sample, and a negative control sample, with a monoclonal antibody that specifically binds to human PD-L1, under conditions that allow for formation of a complex between the antibody or portion thereof and human PD-L1. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In some embodiments, an automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a post primary blocking agent; incubating with NovoLink Polymer; adding a chromogen substrate and developing; and counterstaining with hematoxylin.

For assessing PD-L1 expression in cancer tissue samples, a pathologist may examine the number of membrane PD-L1+ cancer cells in each field under a microscope and mentally estimates the percentage of cells that are positive, then averages them to come to the final percentage. The different staining intensities may be defined as 0/negative, 1+/weak, 2+/moderate, and 3+/strong. Percentage values may be first assigned to the 0 and 3+ buckets, and then the intermediate 1+ and 2+ intensities may be considered. For highly heterogeneous tissues, the specimen may be divided into zones, and each zone may be scored separately and then combined into a single set of percentage values. The percentages of negative and positive cells for the different staining intensities are determined from each area and a median value is given to each zone. A final percentage value may be given to the tissue for each staining intensity category: negative, 1+, 2+, and 3+. The sum of all staining intensities may be 100%.

Staining is also assessed in cancer-infiltrating inflammatory cells such as macrophages and lymphocytes. In most cases macrophages serve as an internal positive control since staining is observed in a large proportion of macrophages. While not required to stain with 3+ intensity, an absence of staining of macrophages may be taken into account to rule out any technical failure. Macrophages and lymphocytes may be assessed for plasma membrane staining and only recorded for all samples as being positive or negative for each cell category. Staining is also characterized according to an outside/inside cancer immune cell designation. “Inside” means the immune cell is within the cancer tissue and/or on the boundaries of the cancer region without being physically intercalated among the cancer cells. “Outside” means that there is no physical association with the cancer, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. In some embodiments, the histoscore may be calculated as follows: Histoscore=[(% cancer×1 (low intensity))+(% cancer×2 (medium intensity))+(% cancer×3 (high intensity)]

In some embodiment, to determine the histoscore, the pathologist may estimate the percentage of stained cells in each intensity category within a specimen. Because expression of most biomarkers is heterogeneous, the histoscore can be a truer representation of the overall expression. The final histoscore range is 0 (no expression) to 300 (maximum expression).

In some embodiments, a means of quantifying PD-L1 expression in a cancer is to determine the adjusted inflammation score (AIS) score defined as the density of inflammation multiplied by the percent PD-L1 expression by cancer-infiltrating inflammatory cells. Taube et al., Colocalization of inflammatory response with B7-hl expression in human melanocytic lesions supports an adaptive resistance mechanism of immune escape, Sci. Transl. Med. 4(127):127ra37 (2012)).

In some embodiments, a means of quantifying PD-L1 expression in a cancer is to determine the Combined Positive Score (CPS), which as described above, is the number of PD-L1 staining cells (tumor cells, lymphocytes, macrophages) divided by the total number of viable tumor cells, multiplied by 100. For some therapeutic treatments, a tumor sample is considered to have PD-L1 expression if CPS≥1. For example, a CPS≥10 is required for a subject to be eligible for certain PD-1 or PD-L1 inhibitor therapies, such as subjects with urothelial cancer (bladder cancer), esophageal squamous cell carcinoma (ESCC), or triple-negative breast cancer being treated with pembrolizumab.

In some embodiments, a means of quantifying PD-L1 expression in a cancer is to determine the Tumor Proportion Score (TPS), which as described above, is the percentage of viable tumor cells showing partial or complete membrane staining at any intensity. For some therapeutic treatments, a tumor sample is considered to have PD-L1 expression if TPS≥1% and high PD-L1 expression if TPS≥50%.

In some embodiments, a means for quantifying PD-L1 expression in a cancer is to determine a Tumor Cell (TC) score. For some therapeutic treatments, a tumor sample is considered to have PD-L1 expression if TC≥50%.

In some embodiments, a means for quantifying PD-L1 expression in a cancer is to determine a Tumor-Infiltrating Immune Cell (IC) score. For some therapeutic treatments, a tumor sample is considered to have PD-L1 expression if a specimen contains PD-L1 staining of any intensity in tumor infiltrating immune cells occupying ≥5% of tumor area.

In some embodiments, a means of quantifying PD-L1 expression in a cancer is the Agilent (Dako) PD-L1 IHC 223 pharmDx Assay™, a description of which may be found in at least one of the following: 1) Physician Labeling, Dako PD-L1 IHC 22C3 pharmDx, Dako North America, Inc., Carpinteria, CA; 2) Keytruda package insert (2021) Merck & Co., Inc., Kenilworth, NJ; 3) PD-L1 IHC 22C3 pharmDx Instructions for Use (2020) Dako, Agilent Pathology Solutions, Carpinteria, CA; 4) Garon E B, Rizvi N A, Hui R, et al. Pembrolizumab for the treatment of non-small-cell lung cancer, N. Engl. J. Med. 372(21):2018-2028 (2015); and 5) Roach C, Zhang N, Corigliano E, et al. Development of a companion diagnostic PD-L1 immunohistochemistry assay for pembrolizumab therapy in non-small-cell lung cancer, Appl Immunohistochem Mol. Morphol. 24:392-397 (2016).

In some embodiments, a means of quantifying PD-L1 expression in a cancer is the Agilent (Dako) PD-L1 IHC 28-8 pharmDx Assay™, a description of which may be found in at least one of the following: 1) Physician Labeling, Dako PD-L1 IHC 28-8 pharmDx (2020) Dako North America, Inc., Carpinteria, CA; 2) OPDIVO package insert (2021) Bristol Myers Squibb, New York, NY; 3) PD-L1 IHC 28-8 pharm Dx: Interpretation Manual (2021), Dako, Agilent Pathology Solutions, Carpinteria, CA; and 4) Phillips T, Simmons P, Inzunza H D, Cogswell J, Novotny J Jr, Taylor C, et al. Development of an automated PD-L1 immunohistochemistry (IHC) assay for non-small cell lung cancer, Appl. Immunohistochem. Mol. Morphol. 23:541-9 (2015).

In some embodiments, a means of quantifying PD-L1 expression in a cancer is the Agilent (Dako) PD-L1 IHC 73-10 Assay™, a description of which may be found in at least one of the following: 1) Hans, J. G. et al. PD-L1 Immunohistochemistry Assay Comparison Studies in Non-Small Cell Lung Cancer: Characterization of the 73-10 Assay, J. Thoracic Oncology 15:1306-1316 (2020); and 2) Bavencio package insert (2021) EMD Serono, Inc. Rockland, MA and Pfizer Inc., New York, NY.

In some embodiments, a means of quantifying PD-L1 expression in a cancer is the Ventana PD-L1 (SP142) Assay™, a description of which may be found in at least one of the following: 1) Physician Labeling: Ventana PD-L1 (SP142) Assay (2020) Ventana Medical Systems, Inc. and Roche Diagnostics International, Inc.; 2) Tecentriq package insert (2021) Genentech, Inc., South San Francisco, CA; 3) Ventana PD-L1 (SP142) Assay: Interpretation Guide (2019) Ventana Medical Systems, Inc. and Roche Diagnostics International, Inc.; and 4) Vennapusa et al., Development of a PD-L1 Complementary Diagnostic Immunochemistry Assay (SP142) for Atezolizumab, Appl. Immunohistochem. Mol. Morphol. 27:92-100 (2019).

In some embodiments, a means of quantifying PD-L1 expression in a cancer is the Ventana PD-L1 (SP263) Assay™, a description of which may be found in at least one of the following: 1) Physician Labeling: Ventana PD-L1 (SP263) Assay (2017) Ventana Medical Systems, Inc., Tucson, AZ; 2) Imfinzi package insert (2021), AstraZeneca Pharmaceuticals LP, Wilmington, DE; and 3) Ventana PD-L1 (SP263) Assay Staining: Interpretation Guide (2019) Roche Diagnostics GmbH, Munich, DE.

Table 1 below provides a summary of the above assays, the drugs for which they may be used, and indications for those treatments as currently approved in the US. Some of the combination therapies provided herein utilize the drugs in the indications as listed in Table 1, together with the corresponding assay to determine PD-L1 expression levels.

TABLE 1
Summary of PD-L1 Diagnostic Assays

| Diagnostic-Antibody | Drug | Indication |
|---|---|---|
| 22C3-Dako/Agilent | Pembrolizumab | NSCLC, urothelial, CHL, |
| | KEYTRUDA)-Merck | melanoma, HNSCC, gastric/GEJ |
| 28-8-Dako/Agilent | Nivolumab (OPDIVO)- | CRC, melanoma, nsNSCLC, renal |
| | Bristol Myers Squibb | cancer, advanced liver, renal and |
| | | urothelical cancers, HNSCC, |
| 73-10-Dako/Agilent | Avelumab (BAVENCIO)- | Urothelial cancer, merkel cell |
| | EMD Serono/Pfizer | carcinoma |
| SP142-Ventana | Atezolizumab | NSCLC, urothelial cancer |
| | (TECENTRIQ)-Genentech | |
| SP263-Ventana | Durvalumab (IMFINZI)- | Advanced NSCLC, urothelial |
| | AstraZeneca | cancer |

Additionally, O'Malley et al., Immunohistochemical detection of PD-L1 among diverse human neoplasms in a reference laboratory: observations based upon 62,896 cases, Modern Pathology 32:929-942 (2019), provides a description evaluating PD-L1 expression using antibody clones 22C3, 28-8, SP142, or SP263, in various types of cancers.

### IV. Exemplary Antibodies

### Exemplary PD-1 Inhibitors and PD-L1 Inhibitors

In certain embodiments, the methods provided herein comprise administering a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, methods provided herein comprise administering a PD-1 inhibitor. In some embodiments, the PD-1 inhibitor is an anti-PD-1 antibody. In some embodiments, the anti-PD-1 antibody is AMP-224, CT-011, cemiplimab, camrelizumab, sintilimab, tislelizumab, TSR-042, PDR001, toripalimab, BGB-A317, nivolumab (also known as ONO-4538, BMS-936558, or MDX1106), pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab. In another embodiment, the PD-1 antibody is camrelizumab. In a further embodiment, the PD-1 antibody is sintilimab. In some embodiments, the PD-1 antibody is tislelizumab. In certain embodiments, the PD-1 antibody is TSR-042. In yet another embodiment, the PD-1 antibody is PDR001. In yet another embodiment, the PD-1 antibody is toripalimab.

In certain embodiments, methods provided herein comprises administering a PD-L1 inhibitor. In some embodiments, the PD-L1 inhibitor is an anti-PD-L1 antibody. In some embodiments, the anti-PD-L1 antibody is MEDI4736 (also known as durvalumab or IMFINZI®), BMS-936559 (also known as MDX-1105-01), atezolizumab (also known as MPDL3280A, and Tecentriq®), or avelumab (also known as BAVENCIO®). In one embodiment, the anti-PD-L1 antibody is MEDI4736 (durvalumab). In another embodiment, the anti-PD-L1 antibody is BMS-936559. In yet another embodiment, the PD-L1 inhibitor is atezolizumab. In a further embodiment, the PD-L1 inhibitor is avelumab.

### Exemplary Anti-TIGIT Antibodies

The anti-TIGIT antibodies utilized in certain of the treatment methods described herein have various activities. For example, in some embodiments, the anti-TIGIT antibody inhibits interaction between TIGIT and one or both of the ligands CD155 and CD112. In some embodiments, the anti-TIGIT antibody inhibits the interaction between TIGIT and CD155 in a functional bioassay, allowing CD155-CD226 signaling to occur.

The present inventors found that, surprisingly, anti-TIGIT antibodies exhibit synergy with PD-1/PD-L1 blockade even in PD-L1 low cancers. As demonstrated herein, administering an anti-TIGIT antibody in combination with an anti-PD-1 and/or anti-PD-L1 antibody to a mouse model comprising a cancer that expresses a low level of PD-L1 results in reduction of tumor size and/or growth rate.

In certain embodiments, the anti-TIGIT antibody is MTIG7192A or a nonfucosylated version thereof. In another embodiment, the anti-TIGIT antibody is BMS-986207 or a nonfucosylated version thereof. In yet another embodiment, the anti-TIGIT antibody is OMP-313M32 or a nonfucosylated version thereof. In one embodiment, the TIGIT inhibitor is MK-7684 or a nonfucosylated version thereof. In another embodiment, the anti-TIGIT antibody is AB154 or a nonfucosylated version thereof. In yet another embodiment, the anti-TIGIT antibody is CGEN-15137 or a nonfucosylated version thereof. In one embodiment, the anti-TIGIT antibody is SEA-TGT. In another embodiment, the anti-TIGIT antibody is ASP8374 or a nonfucosylated version thereof. In yet another embodiment, the anti-TIGIT antibody is AJUD008 or a nonfucosylated version thereof.

In some embodiments, an anti-TIGIT antibody, such as a nonfucosylated anti-TIGIT antibody, binds to human TIGIT protein or a portion thereof with high affinity. In some embodiments, the antibody has a binding affinity (K_{D}) for human TIGIT of less than 5 nM, less than 1 nM, less than 500 pM, less than 250 pM, less than 150 pM, less than 100 pM, less than 50 pM, less than 40 pM, less than 30 pM, less than 20 pM, or less than about 10 pM. In some embodiments, the antibody has a binding affinity (K_{D}) for human TIGIT of less than 50 pM. In some embodiments, the antibody has a K_{D} for human TIGIT in the range of about 1 pM to about 5 nM, e.g., about 1 pM to about 1 nM, about 1 pM to about 500 pM, about 5 pM to about 250 pM, or about 10 pM to about 100 pM.

In some embodiments, in addition to binding to human TIGIT with high affinity, a nonfucosylated anti-TIGIT antibody exhibits cross-reactivity with cynomolgus monkey (“cyno”) TIGIT and/or mouse TIGIT. In some embodiments, the anti-TIGIT antibody binds to mouse TIGIT with a binding affinity (K_{D}) of 100 nM or less. In some embodiments, the anti-TIGIT antibody binds to human TIGIT with a K_{D} of 5 nM or less, and cross-reacts with mouse TIGIT with a K_{D} of 100 nM or less. In some embodiments, an anti-TIGIT antibody that binds to a human TIGIT also exhibits cross-reactivity with both cynomolgus monkey TIGIT and mouse TIGIT.

In some embodiments, antibody cross-reactivity is determined by detecting specific binding of the anti-TIGIT antibody to TIGIT that is expressed on a cell (e.g., a cell line that expresses human TIGIT, cynomolgus monkey TIGIT, or mouse TIGIT, or a primary cell that endogenously expresses TIGIT, e.g., primary T cells that endogenously express human TIGIT, cyno TIGIT, or mouse TIGIT). In some embodiments, antibody binding and antibody cross-reactivity is determined by detecting specific binding of the anti-TIGIT antibody to purified or recombinant TIGIT (e.g., purified or recombinant human TIGIT, purified or recombinant cyno TIGIT, or purified or recombinant mouse TIGIT) or a chimeric protein comprising TIGIT (e.g., an Fc-fusion protein comprising human TIGIT, cynomolgus monkey TIGIT, or mouse TIGIT, or a His-tagged protein comprising human TIGIT, cyno TIGIT, or mouse TIGIT).

In some embodiments, the anti-TIGIT antibodies provided herein inhibit interaction between TIGIT and the ligand CD155. In some embodiments, the anti-TIGIT antibodies provided herein inhibit interaction between TIGIT and the ligand CD112. In some embodiments, the anti-TIGIT antibodies provided herein inhibit interaction between TIGIT and both of the ligands CD155 and CD112.

In some embodiments, an anti-TIGIT antibody that binds to human TIGIT comprises a light chain variable region sequence, or a portion thereof, and/or a heavy chain variable region sequence, or a portion thereof, derived from any of the following antibodies described herein: Clone 13, Clone 13A, Clone 13B, Clone 13C, or Clone 13D. The amino acid sequences of the CDR, light chain variable domain (VL), and heavy chain variable domain (VH) of the anti-TIGIT antibodies Clone 13, Clone 13A, Clone 13B, Clone 13C, and Clone 13D are set forth in the Table of Sequences below.

In some embodiments, an anti-TIGIT antibody comprises one or more (e.g., one, two, three, four, five, or six) of:
a heavy chain CDR1 sequence comprising an amino acid sequence selected from SEQ ID NO:7, SEQ ID NO:8, and SEQ ID NO:9;
a heavy chain CDR2 sequence comprising an amino acid sequence selected from SEQ ID NO:10, SEQ ID NO:11, SEQ ID NO:12, and SEQ ID NO:13;
a heavy chain CDR3 sequence comprising an amino acid sequence selected from SEQ ID NO:14, SEQ ID NO:15 and 16;
a light chain CDR1 sequence comprising an amino acid sequence of SEQ ID NO:17;
a light chain CDR2 sequence comprising an amino acid sequence of SEQ ID NO:18; and/or
a light chain CDR3 sequence comprising the amino acid sequence of SEQ ID NO:19.

In some embodiments, an anti-TIGIT antibody comprises a heavy chain CDR1 sequence comprising the amino acid sequence of SEQ ID NO: 7, SEQ ID NO:8, or SEQ ID NO:9; a heavy chain CDR2 sequence comprising the amino acid sequence of SEQ ID NO:10, SEQ ID NO:11, SEQ ID NO:12, or SEQ ID NO:13; and a heavy chain CDR3 sequence comprising the amino acid sequence of SEQ ID NO: 14, SEQ ID NO:15, or 16.

In some embodiments, an anti-TIGIT antibody comprises a light chain CDR1 sequence comprising the amino acid sequence of SEQ ID NO:17; a light chain CDR2 sequence comprising the amino acid sequence of SEQ ID NO:18; and a light chain CDR3 sequence comprising the amino acid sequence of SEQ ID NO:19.

In some embodiments, an anti-TIGIT antibody comprises a heavy chain CDR1 sequence comprising the amino acid sequence of SEQ ID NO: 7, SEQ ID NO:8, or SEQ ID NO:9; a heavy chain CDR2 sequence comprising the amino acid sequence of SEQ ID NO:10, SEQ ID NO:11, SEQ ID NO:12, or SEQ ID NO:13; a heavy chain CDR3 sequence comprising the amino acid sequence of SEQ ID NO:14, SEQ ID NO:15, or SEQ ID NO: 16; a light chain CDR1 sequence comprising the amino acid sequence of SEQ ID NO:17; a light chain CDR2 sequence comprising the amino acid sequence of SEQ ID NO:18; and a light chain CDR3 sequence comprising the amino acid sequence of SEQ ID NO:19.

In some embodiments, an anti-TIGIT antibody comprises a heavy chain CDR1, CDR2, and CDR3, and a light chain CDR1, CDR2, and CDR3 comprising the amino acid sequences of:
(a) SEQ ID NOs: 7, 10, 14, 17, 18, and 19, respectively; or
(b) SEQ ID NOs: 8, 11, 14, 17, 18, and 19, respectively; or
(c) SEQ ID NOs: 9, 12, 15, 17, 18, and 19, respectively; or
(d) SEQ ID NOs: 8, 13, 16, 17, 18, and 19, respectively; or
(e) SEQ ID NOs: 8, 12, 16, 17, 18, and 19, respectively.

In some embodiments, an anti-TIGIT antibody comprises a heavy chain variable region (VH) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:1, SEQ ID NO:2, SEQ ID NO:3, SEQ ID NO:4, or SEQ ID NO:5. In some embodiments, an anti-TIGIT antibody comprises a VH comprising the amino acid sequence of SEQ ID NO:1, SEQ ID NO:2, SEQ ID NO:3, SEQ ID NO:4, or SEQ ID NO:5. In some embodiments, a VH sequence having at least 90% sequence identity to a reference sequence (e.g., SEQ ID NO:1, SEQ ID NO:2, SEQ ID NO:3, SEQ ID NO:4, or SEQ ID NO:5) contains one, two, three, four, five, six, seven, eight, nine, ten or more substitutions (e.g., conservative substitutions), insertions, or deletions relative to the reference sequence but retains the ability to bind to human TIGIT and optionally, retains the ability to block binding of CD155 and/or CD112 to TIGIT.

In some embodiments, an anti-TIGIT antibody comprises a light chain variable region (VL) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:6. In some embodiments, an anti-TIGIT antibody comprises a VL comprising the amino acid sequence of SEQ ID NO:6. In some embodiments, a VL sequence having at least 90% sequence identity to a reference sequence (e.g., SEQ ID NO:6) contains one, two, three, four, five, six, seven, eight, nine, ten or more substitutions (e.g., conservative substitutions), insertions, or deletions relative to the reference sequence but retains the ability to bind to human TIGIT and optionally, retains the ability to block binding of CD155 and/or CD112 to TIGIT.

In some embodiments, an anti-TIGIT antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:1, SEQ ID NO:2, SEQ ID NO:3, SEQ ID NO:4, or SEQ ID NO:5, and comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:6. In some embodiments, an anti-TIGIT antibody comprises a heavy chain variable region comprising the amino acid sequence of SEQ ID NO:1, SEQ ID NO:2, SEQ ID NO:3, SEQ ID NO:4, or SEQ ID NO:5, and comprises a light chain variable region comprising the amino acid sequence of SEQ ID NO:6.

In some embodiments, an anti-TIGIT antibody comprises:
(a) a VH comprising the amino acid sequence of SEQ ID NO:1 and a VL comprising the amino acid sequence of SEQ ID NO:6;
(b) a VH comprising the amino acid sequence of SEQ ID NO:2 and a VL comprising the amino acid sequence of SEQ ID NO:6; or
(c) a VH comprising the amino acid sequence of SEQ ID NO:3 and a VL comprising the amino acid sequence of SEQ ID NO:6; or
(d) a VH comprising the amino acid sequence of SEQ ID NO:4 and a VL comprising the amino acid sequence of SEQ ID NO:6; or
a VH comprising the amino acid sequence of SEQ ID NO:5 and a VL comprising the amino acid sequence of SEQ ID NO:6.

In some embodiments, an anti-TIGIT antibody comprises a heavy chain comprising an amino acid sequence selected from SEQ ID NOs: 20, 21, 22, 23, and 24; and a light chain comprising the amino acid sequence of SEQ ID NO: 25.

In some embodiments, the anti-TIGIT antibody is SEA-TGT, which is a nonfucosylated IgG1 antibody comprising heavy chain CDR1, CDR2, and CDR3, and light chain CDR1, CDR2, and CDR3 comprising the amino acid sequences of SEQ ID NOs: 7, 10, 14, 17, 18, and 19, respectively. The corresponding VH and VL comprise the amino acid sequences of SEQ ID NO:1 and 6, respectively. See, e.g., PCT Publication No. WO 2020/041541.

In some embodiments, an anti-TIGIT antibody for use in the present methods is a nonfucosylated version of an anti-TIGIT antibody disclosed in US 2009/0258013, US 2016/0176963, US 2016/0376365, or WO 2016/028656.

Exemplary Fc Regions with Enhanced Effector Function

In some embodiments, an antibody used in the methods provided herein comprises an Fc that has one or more or all of the following features in any combination: 1) enhanced binding to one or more activating FcγRs, 2) reduced binding to inhibitory FcγRs, 3) is nonfucosylated, 4) has enhanced ADCC activity, 5) has enhanced ADCP activity, 6) activates antigen presenting cells (APCs), 7) enhances CD8 T cell responses, 8) upregulates co-stimulatory receptors, 9) activates an innate cell immune response, and/or 10) engages NK cells. In some embodiments, an anti-TIGIT antibody used in methods provided herein comprises an Fc with one or more of the foregoing features.

Thus, in some embodiments, the anti-TIGIT antibody comprises an Fc with enhanced binding to one or more activating FcγRs and/or reduced binding to one or more inhibitory FcγRs to obtain the desired enhanced FcγR binding profile. Activating FcγRs include one or more of FcγRIIIa, FcγRIIa, and/or FcγRI. Inhibitory FcγRs include, for example, FcγRIIb.

In certain embodiments, the anti-TIGIT antibody comprises an Fc with enhanced binding to at least FcγRIIIa. In other embodiments, the antibody comprises an Fc with enhanced binding to at least FcγRIIIa and FcγRIIa. In some embodiments, the antibody comprises an Fc with enhanced binding to at least FcγRIIIa and FcγRI. In certain embodiments, the antibody comprises an Fc with enhanced binding to FcγRIIIa, FcγRIIa, and FcγRI.

In some embodiments, the anti-TIGIT antibody, in addition to or separately from enhanced binding to an activating FcγR, has reduced binding to one or more inhibitory FcγRs. Thus, in some embodiments, the antibody has reduced binding to FcγRIIa and/or FcγRIIb.

In some embodiments, the anti-TIGIT antibody is nonfucosylated. In some embodiments, the antibody further has one of the FcγR binding profiles described above.

In certain embodiments, the Fc of the anti-TIGIT antibody comprises amino acid changes relative to a wild-type Fc to enhance binding to an activating FcγR, and/or reduce binding to one or more inhibitory FcγRs to obtain an FcγR binding profile such as described above. For example, in some embodiments the Fc of the antibody comprises the substitutions S293D, A330L, and/or I332E in the heavy chain constant region.

Accordingly, anti-TIGIT antibodies used in the methods provided herein may comprise an Fc that has one or more of the following activities: enhanced binding to one or more activating FcγRs; reduced binding to inhibitory FcγRs; enhanced ADCC activity; and/or enhanced ADCP activity. Antibodies having Fc with such activities and the desired activity profile can be generated in a variety of ways, including producing a nonfucosylated protein and/or by engineering the Fc to contain certain mutations that yield the desired activity. Provided herein are certain additional details on methods for generating nonfucosylated antibodies and exemplary engineering approaches.

Antibodies may be glycosylated at conserved positions in their constant regions (Jefferis and Lund, (1997) Chem. Immunol. 65:111-128; Wright and Morrison, (1997) TibTECH The oligosaccharide side chains of the immunoglobulins affect the protein's function (Boyd et al., (1996) Mol. Immunol. 32:1311-1318; Wittwe and Howard, (1990) Biochem. 29:4175-4180), and the intramolecular interaction between portions of the glycoprotein which can affect the conformation and presented three-dimensional surface of the glycoprotein (Jefferis and Lund, supra; Wyss and Wagner, (1996) Current Opin. Biotech. 7:409-416). Oligosaccharides may also serve to target a given glycoprotein to certain molecules based upon specific recognition structures. For example, it has been reported that in agalactosylated IgG, the oligosaccharide moiety “flips” out of the inter-CH2 space and terminal N-acetylglucosamine residues become available to bind mannose binding protein (Malhotra et al., (1995) Nature Med. 1:237-243). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., (1996) Mol. Immunol. 32:1311-1318), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al. (1999) Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Glycosylation variants of antibodies are variants in which the glycosylation pattern of an antibody is altered. Altering means deleting one or more carbohydrate moieties found in the antibody, adding one or more carbohydrate moieties to the antibody, changing the composition of glycosylation (glycosylation pattern), the extent of glycosylation, etc.

Addition of glycosylation sites to the antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of the antibody.

The amino acid sequence is usually altered by altering the underlying nucleic acid sequence. These methods include isolation from a natural source (in the case of naturally-occurring amino acid sequence variants) or preparation by oligonucleotide-mediated (or site-directed) mutagenesis, PCR mutagenesis, and cassette mutagenesis of an earlier prepared variant or a non-variant version of the antibody.

The glycosylation (including glycosylation pattern) of antibodies may also be altered without altering the amino acid sequence or the underlying nucleotide sequence. See, e.g., Pereira et al., 2018, MAbs, 10(5): 693-711. Glycosylation largely depends on the host cell used to express the antibody. Since the cell type used for expression of recombinant glycoproteins, e.g., antibodies, as potential therapeutics is rarely the native cell, significant variations in the glycosylation pattern of the antibodies can be expected. See, e.g., Hse et al., (1997) J. Biol. Chem. 272:9062-9070. In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261; 5,278,299). Glycosylation, or certain types of glycosylation, can be enzymatically removed from the glycoprotein, for example using endoglycosidase H (Endo H). In addition, the recombinant host cell can be genetically engineered, e.g., make defective in processing certain types of polysaccharides. These and similar techniques are known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides

In some embodiments, a form of modification of glycosylation of the anti-TIGIT antibodies is reduced core fucosylation. “Core fucosylation” refers to addition of fucose (“fucosylation”) to N-acetylglucosamine (“GlcNAc”) at the reducing terminal of an N-linked glycan.

A “complex N-glycoside-linked sugar chain” is typically bound to asparagine 297 (according to the number of Kabat). As used herein, the complex N-glycoside-linked sugar chain has a biantennary composite sugar chain, mainly having the following structure:

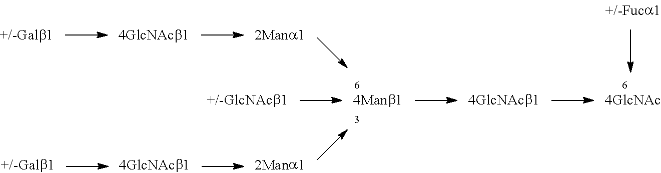

where ± indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has zero, one or more branches of galactose-N-acetylglucosamine (also referred to as “gal-GlcNAc”) and the non-reducing terminal side of gal-GlcNAc optionally has a sialic acid, bisecting N-acetylglucosamine or the like; and 2) a hybrid type, in which the non-reducing terminal side of the core structure has both branches of a high mannose N-glycoside-linked sugar chain and complex N-glycoside-linked sugar chain.

In some methods as provided herein, only a minor amount of fucose is incorporated into the complex N-glycoside-linked sugar chain(s) of the anti-TIGIT antibodies. For example, in various embodiments, less than about 60%, less than about 50%, less than about 40%, less than about 30%, less than about 20%, less than about 15%, less than about 10%, less than about 5%, or less than about 3% of the anti-TIGIT antibodies in a composition have core fucosylation by fucose. In some embodiments, about 2% of the anti-TIGIT antibodies in the composition have core fucosylation by fucose. In various embodiments, when less than 60% of the anti-TIGIT antibodies in a composition have core fucosylation by fucose, the antibodies of the composition may be referred to as “nonfucosylated” or “afucosylated.” In some embodiments, at least 90%, at least 91%, at least 92%, at least 93%, at least 94%, at least 95%, at least 96%, at least 97%, at least 98%, or at least 99% of the anti-TIGIT antibodies in the composition are nonfucosylated.

In certain embodiments, only a minor amount of a fucose analog (or a metabolite or product of the fucose analog) is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 3% of the anti-TIGIT antibodies have core fucosylation by a fucose analog or a metabolite or product of the fucose analog. In some embodiments, about 2% of the anti-TIGIT antibodies have core fucosylation by a fucose analog or a metabolite or product of the fucose analog.

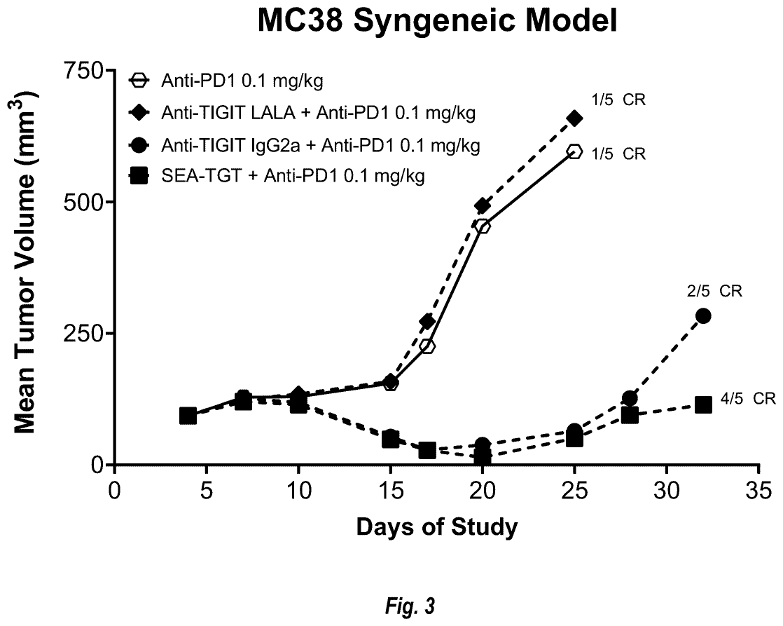

In some embodiments, less that about 60%, less than about 50%, less than about 40%, less than about 30%, less than about 20%, less than about 15%, less than about 10%, less than about 5%, or less than about 3% of the anti-TIGIT antibodies in a composition have a fucose residue on a G0, G1, or G2 glycan structure. (See, e.g., Raju et al., 2012, MAbs 4: 385-391, FIG. 3.) In some embodiments, about 2% of the anti-TIGIT antibodies in the composition have a fucose residue on a G0, G1, or G2 glycan structure. In various embodiments, when less than 60% of the anti-TIGIT antibodies in a composition have a fucose residue on a G0, G1, or G2 glycan structure, the antibodies of the composition may be referred to as “nonfucosylated.” In some embodiments, at least 90%, at least 91%, at least 92%, at least 93%, at least 94%, at least 95%, at least 96%, at least 97%, at least 98%, or at least 99% of the anti-TIGIT antibodies in the composition lack fucose on a G0, G1, or G2 glycan structure. It should be noted that G0 glycans include G0-GN glycans. G0-GN glycans are monoantenary glycans with one terminal GlcNAc residue. G1 glycans include G1-GN glycans. G1-GN glycans are monoantenary glycans with one terminal galactose residue. G0-GN and G1-GN glycans can be fucosylated or nonfucosylated.

A variety of methods for generating nonfucosylated antibodies can be utilized. Exemplary strategies include the use of cell lines lacking certain biosynthetic enzymes involved in fucosylation pathways or the inhibition or the knockout of certain genes involved in the fucosylation pathway. A review of such approaches is provided by Pereira, et al. (2018) mAbs 10:693-711, which is incorporated herein by reference in its entirety.

For example, methods of making nonfucosylated antibodies, such as the nonfucosylated anti-TIGIT antibodies disclosed herein, by incubating antibody-producing cells with a fucose analogue are described, e.g., in WO2009/135181 and U.S. Pat. No. 8,163,551. Briefly, cells that have been engineered to express the antibodies are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In one embodiment, the fucose analogue is 2-flurofucose. Methods of using fucose analogues in growth medium and other fucose analogues are disclosed, e.g., in WO 2009/135181, which is herein incorporated by reference.

Other methods for engineering cell lines to reduce core fucosylation included gene knock-outs, gene knock-ins and RNA interference (RNAi). See, e.g., Pereira et al., 2018, mAbs, 10(5):693-711. In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely.

Other strategies that may be used include GlycoMAb® (U.S. Pat. No. 6,602,684) and Potelligent⁻ (BioWa).

Any of these methods can be used to generate a cell line that would be able to produce a nonfucosylated antibody.

Various engineering approaches can also be utilized to obtain Fc regions with the desired FcγR activity and effector function. In some embodiments, the Fc is engineered to have the following combination of mutations: S239D, A330L and 1332E, which increases the affinity of the Fc domain for FcγRIIIA and consequently increases ADCC. Additional substitutions that enhance affinity for FcγRIIIa include, for example, T256A, K290A, S298A, E333A, and K334A. Substitutions that enhance binding to activating FcγRIIIa and reduced binding to inhibitory FcγRIIIb include, for example, F243L/R292P/Y300L/V305I/P396L and F243L/R292P/Y300L/L235V/P396L. In some embodiments, the substitutions are in an IgG1 Fc backbone.

Oligosaccharides covalently attached to the conserved Asn297 are involved in the ability of the Fc region of an IgG to bind FcγR (Lund et al., 1996, J Immunol. 157:4963-69; Wright and Morrison, 1997, Trends Biotechnol. 15:26-31). Engineering of this glycoform on IgG can significantly improve IgG-mediated ADCC. Addition of bisecting N-acetylglucosamine modifications (Umana et al., 1999, Nat. Biotechnol. 17:176-180; Davies et al., 2001, Biotech. Bioeng. 74:288-94) to this glycoform or removal of fucose (Shields et al., 2002, J. Biol. Chem. 277:26733-40; Shinkawa et al., 2003, J. Biol. Chem. 278:6591-604; Niwa et al., 2004, Cancer Res. 64:2127-33) from this glycoform are two examples of IgG Fc engineering that improves the binding between IgG Fc and FcγR, thereby enhancing Ig-mediated ADCC activity.

A systemic substitution of solvent-exposed amino acids of human IgG1 Fc region has generated IgG variants with altered FcγR binding affinities (Shields et al., 2001, J. Biol. Chem. 276:6591-604). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., 2001, J. Biol. Chem. 276:6591-604; Okazaki et al., 2004, J. Mol. Biol. 336:1239-49).

Many methods are available to determine the amount of fucosylation on an antibody. Methods include, e.g., LC-MS via PLRP-S chromatography, electrospray ionization quadrupole TOF MS, Capillary Electrophoresis with Laser-Induced Fluorescence (CE-LIF), and Hydrophilic Interaction Chromatography with Fluorescence Detection (HILIC).

### Preparation of Antibodies

For preparing an antibody, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)).

The genes encoding the heavy and light chains of an antibody of interest can be cloned from a cell, e.g., the genes encoding a monoclonal antibody can be cloned from a hybridoma that expresses the antibody and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. (2010) PEDS 23:311; and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can also be adapted to produce antibodies. Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or antibodies covalently bound to immunotoxins (see, e.g., U.S. Pat. No. 4,676,980, WO 91/00360; and WO 92/200373).

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a heavy chain and light chain, the heavy chain and heavy chain and light chain may be expressed using a single vector, e.g., in a di-cistronic expression unit, or be under the control of different promoters. In other embodiments, the heavy chain and light chain region may be expressed using separate vectors. Heavy chains and light chains as described herein may optionally comprise a methionine at the N-terminus.

In some embodiments, antibody fragments (such as a Fab, a Fab′, a F(ab′)₂, a scFv, or a diabody) are generated. Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870.

In some embodiments, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007).

In some embodiments, multispecific antibodies are provided, e.g., a bispecific antibody. Multispecific antibodies are antibodies that have binding specificities for at least two different antigens or for at least two different epitopes of the same antigen. Methods for making multispecific antibodies include, but are not limited to, recombinant co-expression of two pairs of heavy chain and light chain in a host cell (see, e.g., Zuo et al., Protein Eng Des Sel, 2000, 13:361-367); “knobs-into-holes” engineering (see, e.g., Ridgway et al., Protein Eng Des Sel, 1996, 9:617-721); “diabody” technology (see, e.g., Hollinger et al., PNAS (USA), 1993, 90:6444-6448); and intramolecular trimerization (see, e.g., Alvarez-Cienfuegos et al., Scientific Reports, 2016, doi:/10.1038/srep28643); see also, Spiess et al., Molecular Immunology, 2015, 67(2), Part A:95-106.

### Selection of Constant Region

Heavy and light chain variable regions of the antibodies described herein can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human IgG4 lacks complement-dependent cytotoxicity. Human IgG1 and IgG3 also induce stronger cell mediated effector functions than human IgG2 and IgG4. Light chain constant regions can be lambda or kappa. Antibodies can be expressed as tetramers containing two light and two heavy chains, as separate heavy chains, light chains, as Fab, Fab′, F(ab′)2, and Fv, or as single chain antibodies in which heavy and light chain variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of one or more other isotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

For constructing desired antibodies, in some embodiments, exemplary substitution include the amino acid substitution of the native amino acid to a cysteine residue is introduced at amino acid position 234, 235, 237, 239, 267, 298, 299, 326, 330, or 332, preferably an S239C mutation in a human IgG1 isotype (numbering is according to the EU index (Kabat, Sequences of Proteins of Immunological Interest (National Institutes of Health, Bethesda, M D, 1987 and 1991); see US 20100158909, which is herein incorporated reference). The presence of an additional cysteine residue may allow interchain disulfide bond formation. Such interchain disulfide bond formation can cause steric hindrance, thereby reducing the affinity of the Fc region-FcγR binding interaction. Other substitutions at any of positions 234, 235, 236 and/or 237 reduce affinity for Fcγ receptors, particularly FcγRI receptor (see, e.g., U.S. Pat. Nos. 6,624,821, 5,624,821).

The in vivo half-life of an antibody can also impact its effector functions. The half-life of an antibody can be increased or decreased to modify its therapeutic activities. FcRn is a receptor that is structurally similar to MHC Class I antigen that non-covalently associates with β2-micro g lobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG1 involved in FcRn binding has been mapped (Shields et al., 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al., 2001, J Biol. Chem. 276:6591-604). IgG1 molecules harboring these substitutions have longer serum half-lives. Consequently, these modified IgG1 molecules may be able to carry out their effector functions, and hence exert their therapeutic efficacies, over a longer period of time compared to unmodified IgG1. Other exemplary substitutions for increasing binding to FcRn include a Gln at position 250 and/or a Leu at position 428. EU numbering is used for all positions in the constant region.

Complement fixation activity of antibodies (both C1q binding and CDC activity) can be improved by substitutions at Lys326 and Glu333 (Idusogie et al., 2001, J. Immunol. 166:2571-2575). The same substitutions on a human IgG2 backbone can convert an antibody isotype that binds poorly to C1q and is severely deficient in complement activation activity to one that can both bind C1q and mediate CDC (Idusogie et al., 2001, J. Immunol. 166:2571-75). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., 1995, J. Immunol. 154:2226-36). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., 1992, J. Immunol. 148:2918-22). In addition, a bispecific diabody construct with specificity for C1q also confers CDC activity (Kontermann et al., 1997, Nat. Biotech. 15:629-31).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity but only slightly reduces (about three-fold weaker) affinity for C1q. This alteration destroys the glycosylation site and the presence of carbohydrate that is required for complement activation. Any other substitution at this site also destroys the glycosylation site. The following mutations and any combination thereof also reduce C1q binding: D270A, K322A, P329A, and P311S (see WO 06/036291).

Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRN.

### Nucleic Acids, Vectors, and Host Cells

In some embodiments, the antibodies described herein are prepared using recombinant methods. Accordingly, in some aspects, the invention provides isolated nucleic acids comprising a nucleic acid sequence encoding any of the antibodies described herein (e.g., any one or more of the CDRs described herein); vectors comprising such nucleic acids; and host cells into which the nucleic acids are introduced that are used to replicate the antibody-encoding nucleic acids and/or to express the antibodies. In some embodiments, the host cell is eukaryotic, e.g., a Chinese Hamster Ovary (CHO) cell; or a human cell.

In some embodiments, a polynucleotide (e.g., an isolated polynucleotide) comprises a nucleotide sequence encoding an antibody described herein. In some embodiments, the polynucleotide comprises a nucleotide sequence encoding one or more amino acid sequences (e.g., CDR, heavy chain, light chain, and/or framework regions) disclosed herein. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence (e.g., a CDR, heavy chain, light chain, or framework region sequence) disclosed herein.

In a further aspect, methods of making an antibody described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. Cloning vectors are available from commercial vendors such as BioRad, Stratagene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

### Expression of Recombinant Antibodies

Antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

Mammalian cells are a preferred host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NS0. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148:1149 (1992).

Once expressed, antibodies can be purified according to standard procedures of the art, including HPLC purification, column chromatography, gel electrophoresis and the like (see generally, Scopes, Protein Purification (Springer-Verlag, NY, 1982)).

### Antibody Characterization

Methods for analyzing binding affinity, binding kinetics, and cross-reactivity are known in the art. See, e.g., Ernst et al., Determination of Equilibrium Dissociation Constants, Therapeutic Monoclonal Antibodies (Wiley & Sons ed. 2009). These methods include, but are not limited to, solid-phase binding assays (e.g., ELISA assay), immunoprecipitation, surface plasmon resonance (SPR, e.g., Biacore™ (GE Healthcare, Piscataway, NJ)), kinetic exclusion assays (e.g. KinExA®), flow cytometry, fluorescence-activated cell sorting (FACS), BioLayer interferometry (e.g., Octet™ (FortéBio, Inc., Menlo Park, CA)), and Western blot analysis. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given wavelength is shined on the surface, the changes to the optical reflectivity of the gold indicate binding, and the kinetics of binding. In some embodiments, kinetic exclusion assays are used to determine affinity. This technique is described, e.g., in Darling et al., Assay and Drug Development Technologies Vol. 2, number 6 647-657 (2004). In some embodiments, BioLayer interferometry assays are used to determine affinity. This technique is described, e.g., in Wilson et al., Biochemistry and Molecular Biology Education, 38:400-407 (2010); Dysinger et al., J. Immunol. Methods, 379:30-41 (2012).

### V. Therapeutic Methods

In some embodiments, methods for treating cancer in a subject are provided. In some embodiments, the methods comprise administering to a subject with cancer (1) an anti-TIGIT antibody, and (2) an anti-PD-1 antibody or an anti-PD-L1 antibody, wherein the anti-TIGIT antibody comprises an Fc region with enhanced effector function.

In some embodiments, the methods are based in part on the surprising finding that cancers expressing low levels of PD-L1 can be treated with an anti-TIGIT antibody in combination with an anti-PD-1 antibody and/or an anti-PD-L1 antibody. This synergy between an anti-TIGIT antibody and an anti-PD-1 antibody and/or an anti-PD-L1 antibody enables treatment of cancers for which there currently are no approved monotherapies using anti-PD1 antibodies or PD-L1 antibodies.

For example, Table 2 shows therapeutic dosing level, PD-L1 expression level, and mutation status for treatments of certain cancers with certain anti-PD-1 antibodies. Table 3 shows therapeutic dosing level, PD-L1 expression level, and mutation status for treatments of certain cancers with certain anti-PD-L1 antibodies. As can be seen from these tables, many such antibodies are not approved for subjects having cancer expressing a PD-L1 level below certain thresholds, and are not approved for subjects having cancer comprising certain mutations. As described in greater detail below, the methods provided herein can be used to treat patients with tumors expressing PD-L1 at levels below the approved cut-off, or threshold, levels, patients having mutations such as those listed in the table that make them less responsive to treatment with an anti-PD1 or anti-PD-L1 antibody, and/or to treat the patients with a dose of an anti-PD1 or anti-PD-L1 antibody below the approved dose as listed in the table.

For example, while in some embodiments, the anti-PD-1 antibody and/or anti-PD-L1 antibody is administered at a therapeutic dose, such as a dose described in Table 2 and/or Table 3, in other embodiments, the anti-PD-1 antibody and/or anti-PD-L1 antibody can be administered at a sub-therapeutic dose, such as a dose that is lower than and/or that is administered less frequently than a dose described in Table 2 and/or Table 3. In some embodiments, the method treats a subject who has cancer comprising mutations that result in exclusion of the subject from certain treatments, such as the treatments described in Table 2 and/or Table 3.

As such, in some embodiments, the methods disclosed herein provide treatment of a subject with cancer for whom the treatments described in Table 2 and/or Table 3 are unavailable. These methods are discussed below in greater detail, such as regarding thresholds for PD-L1 level and dosing.

TABLE 2
Therapeutic dosing of certain anti-PD-1 antibodies

| PD-1 | Indication | PD-L1 cutoff | Mutations | Dose |
|---|---|---|---|---|
| KEYTRUDA | Melanoma | | | 200 mg every 3 weeks or |
| (pembrolizumab) | | | | 400 mg every 6 weeks |
| US Label dated | Non-Small Cell Lung | as a single agent for the first-line | in combination with | 200 mg every 3 weeks or |
| 2021 Mar. 21 | Cancer (NSCLC) | treatment of patients with NSCLC | pemetrexed and | 400 mg every 6 weeks. |
| Administer | | expressing PD-L1 [Tumor | platinum chemotherapy, | |
| KEYTRUDA as an | | Proportion Score (TPS) ≥1%] as | as first-line treatment | |
| intravenous infusion | | determined by an FDA-approved | of patients with | |
| over 30 minutes | | test, with no EGFR or ALK | metastatic nonsquamous | |
| Injection: 100 mg/ | | genomic tumor aberrations, and | NSCLC, with no EGFR | |
| 4 mL (25 mg/mL) | | is: | or ALK genomic | |
| solution in a single- | | stage III where patients are not | tumor aberrations. | |
| dose vial | | candidates for surgical resection or | | |
| | | definitive chemoradiation, or | | |
| | | metastatic. | | |
| | | as a single agent for the treatment | | |
| | | of patients with metastatic NSCLC | | |
| | | whose tumors express PD-L1 | | |
| | | (TPS ≥1%) as determined by an | | |
| | | FDA-approved test, with disease | | |
| | | progression on or after platinum- | | |
| | | containing chemotherapy. | | |
| | | Patients with EGFR or ALK | | |
| | | genomic tumor aberrations | | |
| | | should have disease progression | | |
| | | on FDA-approved therapy for | | |
| | | these aberrations prior to | | |
| | | receiving KEYTRUDA. | | |
| | Small Cell Lung Cancer | | | 200 mg every 3 weeks or |
| | (SCLC) | | | 400 mg every 6 weeks. |
| | Head and Neck | as a single agent for the first-line | | 200 mg every 3 weeks or |
| | Squamous Cell Cancer | treatment of patients with metastatic | | 400 mg every 6 weeks |
| | (HNSCC) | or with unresectable, recurrent | | |
| | | HNSCC whose tumors express PD- | | |
| | | L1 [Combined Positive Score | | |
| | | (CPS) ≥1] as determined by an | | |
| | | FDA-approved test | | |
| | Classical Hodgkin | | | 200 mg every 3 weeks or |
| | Lymphoma (cHL) | | | 400 mg every 6 weeks for |
| | | | | adults; 2 mg/kg (up to 200 |
| | | | | mg) every 3 weeks for |
| | | | | pediatrics |
| | Primary Mediastinal | | | 200 mg every 3 weeks or |
| | Large B-Cell | | | 400 mg every 6 weeks for |
| | Lymphoma (PMBCL) | | | adults; 2 mg/kg (up to 200 |
| | | | | mg) every 3 weeks for |
| | | | | pediatrics |
| | Urothelial Carcinoma | for the treatment of patients with | | 200 mg every 3 weeks or |
| | | locally advanced or metastatic | | 400 mg every 6 weeks |
| | | urothelial carcinoma who are not | | |
| | | eligible for cisplatin-containing | | |
| | | chemotherapy and whose tumors | | |
| | | express PD-L1 [Combined | | |
| | | Positive Score (CPS) ≥10] as | | |
| | | determined by an FDA-approved | | |
| | | test, or in patients who are not | | |
| | | eligible for any platinum-containing | | |
| | | chemotherapy regardless of PD-L1 | | |
| | | status | | |
| | Microsatellite Instability- | | | 200 mg every 3 weeks or |
| | High or Mismatch Repair | | | 400 mg every 6 weeks for |
| | Deficient Cancer | | | adults; 2 mg/kg (up to 200 |
| | | | | mg) every 3 weeks for |
| | | | | pediatrics |
| | Microsatellite Instability- | | | 200 mg every 3 weeks or |
| | High or Mismatch Repair | | | 400 mg every 6 weeks |
| | Deficient Colorectal | | | |
| | Cancer (CRC) | | | |
| | Gastric Cancer | for the treatment of patients with | | 200 mg every 3 weeks or |
| | | recurrent locally advanced or | | 400 mg every 6 weeks |
| | | metastatic gastric or | | |
| | | gastroesophageal junction | | |
| | | adenocarcinoma whose tumors | | |
| | | express PD-L1 [Combined | | |
| | | Positive Score (CPS) ≥1] as | | |
| | | determined by an FDA-approved | | |
| | | test, with disease progression on or | | |
| | | after 2 or more prior lines of | | |
| | | therapy including fluoropyrimidine- | | |
| | | and platinum-containing | | |
| | | chemotherapy and if appropriate, | | |
| | | HER2/neu-targeted therapy | | |
| | Esophageal Cancer | for the treatment of patients with | | 200 mg every 3 weeks or |
| | | locally advanced or metastatic | | 400 mg every 6 weeks |
| | | esophageal or gastroesophageal | | |
| | | junction (GEJ) (tumors with | | |
| | | epicenter 1 to 5 centimeters above | | |
| | | the GEJ) carcinoma that is not | | |
| | | amenable to surgical resection or | | |
| | | definitive chemoradiation | | |
| | | either: | | |
| | | in combination with platinum- | | |
| | | and fluoropyrimidine-based | | |
| | | chemotherapy, or | | |
| | | as a single agent after one or more | | |
| | | prior lines of systemic therapy for | | |
| | | patients with tumors of squamous | | |
| | | cell histology that express PD-L1 | | |
| | | (CPS ≥10) as determined by an | | |
| | | FDA-approved test (1.11, 2.1). | | |
| | Cervical Cancer | for the treatment of patients with | | 200 mg every 3 weeks or |
| | | recurrent or metastatic cervical | | 400 mg every 6 weeks |
| | | cancer with disease progression on | | |
| | | or after chemotherapy whose | | |
| | | tumors express PD-L1 [Combined | | |
| | | Positive Score (CPS) ≥1] as | | |
| | | determined by an FDA-approved | | |
| | | test. | | |
| | Hepatocellular Carcinoma | | | 200 mg every 3 weeks or |
| | (HCC) | | | 400 mg every 6 weeks |
| | Merkel Cell Carcinoma | | | 200 mg every 3 weeks or |
| | (MCC) | | | 400 mg every 6 weeks for |
| | | | | adults; 2 mg/kg (up to 200 |
| | | | | mg) every 3 weeks for |
| | | | | pediatrics. |
| | Renal Cell Carcinoma | | | 200 mg every 3 weeks or |
| | (RCC) | | | 400 mg every 6 weeks |
| | | | | with axitinib 5 mg orally |
| | | | | twice daily. |
| | Endometrial Carcinoma | | | 200 mg every 3 weeks or |
| | | | | 400 mg every 6 weeks |
| | | | | with lenvatinib 20 mg |
| | | | | orally once daily for |
| | | | | tumors that are not MSI-H |
| | | | | or dMMR |
| | Tumor Mutational | | | 200 mg every 3 weeks or |
| | Burden-High (TMB-H) | | | 400 mg every 6 weeks for |
| | Cancer | | | adults; 2 mg/kg (up to 200 |
| | | | | mg) every 3 weeks for |
| | | | | pediatrics |
| | Cutaneous Squamous Cell | | | 200 mg every 3 weeks or |
| | Carcinoma (cSCC) | | | 400 mg every 6 weeks |
| | Triple-Negative Breast | in combination with chemotherapy, | | 200 mg every 3 weeks or |
| | Cancer (TNBC) | for the treatment of | | 400 mg every 6 weeks |
| | | patients with locally recurrent | | |
| | | unresectable or metastatic | | |
| | | TNBC whose tumors express PD- | | |
| | | L1 [Combined Positive Score | | |
| | | (CPS) ≥10] as determined by an | | |
| | | FDA approved test. | | |
| | Adult Indications: | | | |
| | Additional Dosing | | | |
| | Regimen of 400 mg Every | | | |
| | 6 Weeks (for use at an | | | |
| | additional recommended | | | |
| | dosage of 400 mg every 6 | | | |
| | weeks for all approved | | | |
| | adult indications) | | | |
| OPDIVO | Melanoma | | | Unresectable or metastatic |
| (nivolumab) | | | | melanoma |
| US Label dated | | | | 240 mg every 2 weeks or |
| 2021 Jan. 22 | | | | 480 mg every 4 weeks. |
| Administer by | | | | 1 mg/kg followed by |
| intravenous infusion | | | | ipilimumab 3 mg/kg on |
| based upon | | | | the same day every 3 |
| recommended infusion | | | | weeks for 4 doses, then |
| rate for each | | | | 240 mg every 2 weeks or |
| indication. | | | | 480 mg every 4 weeks. |
| Injection: 40 mg/ | | | | Adjuvant treatment of |
| 4 mL, 100 mg/10 mL, | | | | melanoma |
| and 240 mg/24 mL | | | | 240 mg every 2 weeks or |
| solution in a single- | | | | 480 mg every 4 weeks. |
| dose vial. | Non-Small Cell Lung | adult patients with metastatic non-small cell | | 3 mg/kg every 2 weeks |
| | Cancer (NSCLC) | lung cancer expressing PD-L1 (≥1%) as determined | | with ipilimumab 1 mg/kg |
| | | by an FDA-approved test, with no EGFR or | | every 6 weeks. |
| | | ALK genomic tumor aberrations, as first-line | | 360 mg every 3 weeks |
| | | treatment in combination with ipilimumab. (1.3) | | with ipilimumab 1 mg/kg |
| | | adult patients with metastatic or recurrent | | every 6 weeks and 2 |
| | | non-small cell lung cancer with no EGFR or | | cycles of platinum-doublet |
| | | ALK genomic tumor aberrations as first-line | | chemotherapy. |
| | | treatment, in combination with ipilimumab | | 240 mg every 2 weeks or |
| | | and 2 cycles of platinum-doublet | | 480 mg every 4 weeks. |
| | | chemotherapy. (1.3) | | |
| | | patients with metastatic non-small cell lung | | |
| | | cancer and progression on or after platinum- | | |
| | | based chemotherapy. Patients with EGFR or | | |
| | | ALK genomic tumor aberrations should have | | |
| | | disease progression on FDA-approved therapy | | |
| | | for these aberrations prior to receiving OPDIVO. | | |
| | Malignant Pleural | | | 360 mg every 3 weeks |
| | Mesothelioma | | | with ipilimumab 1 mg/kg |
| | | | | every 6 weeks. |
| | Renal Cell Carcinoma | | | 3 mg/kg followed by |
| | (RCC) | | | ipilimumab 1 mg/kg on |
| | | | | the same day every 3 |
| | | | | weeks for 4 doses, then |
| | | | | 240 mg every 2 weeks or |
| | | | | 480 mg every 4 weeks. |
| | | | | 240 mg every 2 weeks or |
| | | | | 480 mg every 4 weeks |
| | | | | administered in |
| | | | | combination with |
| | | | | cabozantinib 40 mg once |
| | | | | daily without food. |
| | | | | 240 mg every 2 weeks or |
| | | | | 480 mg every 4 weeks. |
| | Classical Hodgkin | | | 240 mg every 2 weeks or |
| | Lymphoma (cHL) | | | 480 mg every 4 weeks |
| | Squamous Cell Carcinoma | | | 240 mg every 2 weeks or |
| | of the Head and Neck | | | 480 mg every 4 weeks |
| | (SCCHN) | | | |
| | Urothelial Carcinoma | | | 240 mg every 2 weeks or |
| | | | | 480 mg every 4 weeks. |
| | Colorectal Cancer | | | Adult and pediatric |
| | | | | patients ≥40 kg: 240 mg |
| | | | | every 2 weeks or 480 mg |
| | | | | every 4 weeks. |
| | | | | Pediatric patients <40 |
| | | | | kg: 3 mg/kg every 2 |
| | | | | weeks. |
| | | | | Adult and pediatric |
| | | | | patients ≥40 kg: 3 mg/kg |
| | | | | followed by ipilimumab 1 |
| | | | | mg/kg on the same day |
| | | | | every 3 weeks for 4 doses, |
| | | | | then 240 mg every 2 |
| | | | | weeks or 480 mg every 4 |
| | | | | weeks. |
| | Hepatocellular Carcinoma | | | 240 mg every 2 weeks or |
| | (HCC) | | | 480 mg every 4 weeks. |
| | | | | 1 mg/kg followed by |
| | | | | ipilimumab 3 mg/kg on |
| | | | | the same day every 3 |
| | | | | weeks for 4 doses, then |
| | | | | 240 mg every 2 weeks or |
| | | | | 480 mg every 4 weeks. |
| | Esophageal Squamous | | | 240 mg every 2 weeks or |
| | Cell Carcinoma (ESCC) | | | 480 mg every 4 weeks |
| LIBTAYO | Cutaneous Squamous Cell | | | The recommended dosage |
| (cemiplimab) | Carcinoma (CSCC) | | | of LIBTAYO is 350 mg as |
| US Label dated | Basal Cell Carcinoma | | | an intravenous infusion |
| 2021 Feb. 22 | (BCC) | | | over 30 minutes every 3 |

| | | | |
|---|---|---|---|
| 350 mg/7 mL | Non-Small Cell Lung | for the first-line treatment of patients with | weeks. |
| (50 mg/mL) solution | Cancer (NSCLC) | NSCLC whose tumors have high | |
| in a single-dose vial. | | PD-L1 expression [Tumor Proportion Score | |
| | | (TPS) ≥50%] as determined by an FDA- | |
| | | approved test, with no EGFR, ALK or ROS1 | |
| | | aberrations, and is: | |
| | | locally advanced where patients are not | |
| | | candidates for surgical resection or | |
| | | definitive chemoradiation or metastatic. | |

TABLE 3
Therapeutic dosing of certain anti-PD-L1 antibodies

| PD-L1 | Indication | PD-L1 cutoff | Mutations | Dose |
|---|---|---|---|---|
| TECENTRIQ | Urothelial Carcinoma | for the treatment of adult patients | | Administer TECENTRIQ |
| (atezolizumab) | | with locally advanced or metastatic | | as a single agent as 840 mg |
| US Label dated | | urothelial carcinoma who: | | every 2 weeks, 1200 mg |
| 2021 Feb. 17 | | are not eligible for cisplatin- | | every 3 weeks, or 1680 mg |
| Administer | | containing chemotherapy and | | every 4 weeks. |
| TECENTRIQ | | whose tumors express PD-L1 (PD- | | |
| intravenously over | | L1 stained tumor-infiltrating | | |
| 60 minutes. | | immune cells [IC] covering ≥5% of | | |
| 840 mg/14 mL | | the tumor area), as determined by | | |
| (60 mg/mL) and | | an FDA-approved test, or | | |
| 1200 mg/20 mL | | are not eligible for any platinum- | | |
| (60 mg/mL) solution | | containing chemotherapy regardless | | |
| in a single-dose vial | | of PD-L1 status, or | | |
| | | have disease progression during | | |
| | | or following any platinum- | | |
| | | containing chemotherapy, or within | | |
| | | 12 months of neoadjuvant or | | |
| | | adjuvant chemotherapy. | | |
| | Non-Small Cell Lung | for the first-line treatment of adult patients | | Administer TECENTRIQ |
| | Cancer (NSCLC) | with metastatic NSCLC whose tumors | | as 840 mg every 2 weeks, |
| | | have high PD-L1 expression (PD-L1 | | 1200 mg every 3 weeks, or |
| | | stained ≥50% of tumor cells | | 1680 mg every 4 weeks. |
| | | [TC ≥50%] or PD-L1 stained tumor- | | (2.2) |
| | | infiltrating immune cells | | When administering with |
| | | [IC] covering ≥10% of the tumor area | | chemotherapy with or |
| | | [IC ≥10%] ), as determined by | | without bevacizumab, |
| | | an FDA-approved test, with no EGFR | | administer TECENTRIQ |
| | | or ALK genomic tumor aberrations. | | prior to chemotherapy and |
| | | in combination with bevacizumab, | | bevacizumab when given |
| | | paclitaxel, and carboplatin, for the | | on the same day. |
| | | first-line treatment of adult patients with | | |
| | | metastatic non-squamous NSCLC with | | |
| | | no EGFR or ALK genomic tumor | | |
| | | aberrations. (1.2) | | |
| | | in combination with paclitaxel protein- | | |
| | | bound and carboplatin for the | | |
| | | first-line treatment of adult patients with | | |
| | | metastatic non-squamous NSCLC with | | |
| | | no EGFR or ALK genomic tumor | | |
| | | aberrations (1.2) | | |
| | | for the treatment of adult patients with | | |
| | | metastatic NSCLC who have disease | | |
| | | progression during or following | | |
| | | platinum-containing chemotherapy. | | |
| | | Patients with EGFR or ALK genomic | | |
| | | tumor aberrations should have | | |
| | | disease progression on FDA-approved | | |
| | | therapy for NSCLC harboring these | | |
| | | aberrations prior to receiving | | |
| | | TECENTRIQ | | |
| | Triple-Negative Breast | in combination with paclitaxel | | Administer TECENTRIQ |
| | Cancer (TNBC) | protein-bound for the treatment of | | 840 mg every 2 weeks, |
| | | adult patients with unresectable | | 1200 mg every 3 weeks, or |
| | | locally advanced or metastatic | | 1680 mg every 4 weeks. |
| | | TNBC whose tumors express PD- | | Administer TECENTRIQ |
| | | L1 (PD-L1 stained tumor- | | prior to paclitaxel protein- |
| | | infiltrating immune cells [IC] of | | bound when given on the |
| | | any intensity covering ≥1% of | | same day. For each 28 day |
| | | the tumor area), as determined by | | cycle, paclitaxel protein- |
| | | an FDA approved test. This | | bound is administered at |
| | | indication is approved under | | 100 mg/m2 on days 1, 8, |
| | | accelerated approval based on | | and 15 |
| | | progression free survival. | | |
| | | Continued approval for this | | |
| | | indication may be contingent upon | | |
| | | verification and description of | | |
| | | clinical benefit in a confirmatory | | |
| | | trial(s). | | |
| | Small Cell Lung Cancer | | | Administer TECENTRIQ |
| | (SCLC) | | | as 840 mg every 2 weeks, |
| | | | | 1200 mg every 3 weeks, or |
| | | | | 1680 mg every 4 weeks. |
| | | | | When administering with |
| | | | | carboplatin and etoposide, |
| | | | | administer TECENTRIQ |
| | | | | prior to chemotherapy |
| | | | | when given on the same |
| | | | | day. |
| | Hepatocellular Carcinoma | | | Administer TECENTRIQ |
| | (HCC) | | | as 840 mg every 2 weeks, |
| | | | | 1200 mg every 3 weeks, or |
| | | | | 1680 mg every 4 weeks. |
| | | | | Administer TECENTRIQ |
| | | | | prior to bevacizumab when |
| | | | | given on the same day. |
| | | | | Bevacizumab is |
| | | | | administered at 15 mg/kg |
| | | | | every 3 weeks. |
| | Melanoma | | in combination with | Following completion of a |
| | | | cobimetinib and | 28 day cycle of cobimetinib |
| | | | vemurafenib for the | and vemurafenib, |
| | | | treatment of patients | administer TECENTRIQ |
| | | | with BRAF V600 | 840 mg every 2 weeks, |
| | | | mutation-positive | 1200 mg every 3 weeks, or |
| | | | unresectable or | 1680 mg every 4 weeks |
| | | | metastatic | with cobimetinib 60 mg |
| | | | melanoma. | orally once daily (21 days |
| | | | | on/7 days off) and |
| | | | | vemurafenib 720 mg orally |
| | | | | twice daily. |
| INFINZI | Stage III non-small cell | | | Weight 30 kg and more: |
| (durvalumab) | lung cancer (NSCLC) | | | 10 mg/kg every 2 weeks or |
| US Label dated | | | | 1500 mg every 4 weeks |
| 2021 Feb. 19 | | | | Weight less than 30 kg: |
| Administer IMFINZI | | | | 10 mg/kg every 2 weeks |
| as an intravenous | extensive-stage small cell | | | Weight 30 kg and more: |
| infusion over 60 | lung cancer (ES-SCLC) | | | With etoposide and either |
| minutes. | | | | carboplatin or cisplatin, |
| 500 mg/10 mL (50 | | | | administer IMFINZI 1500 |
| mg/mL) solution or | | | | mg every 3 weeks in |
| 120 mg/2.4 mL (50 | | | | combination with |
| mg/mL) solution in | | | | chemotherapy, and then |
| a single-dose vial | | | | 1500 mg every 4 weeks as a |
| | | | | single agent |
| | | | | Weight less than 30 kg: |
| | | | | With etoposide and either |
| | | | | carboplatin or cisplatin, |
| | | | | administer IMFINZI 20 |
| | | | | mg/kg every 3 weeks in |
| | | | | combination with |
| | | | | chemotherapy, and then 10 |
| | | | | mg/kg every 2 weeks as a |
| | | | | single agent |
| BAVENCIO | Merkel Cell Carcinoma | | | 800 mg every 2 weeks. |
| (avelumab) | (MCC) | | | |
| US Label dated | Urothelial Carcinoma | | | 800 mg every 2 weeks |
| 2020 Nov. 10 | (UC) | | | |
| Administer | Renal Cell Carcinoma | | | 800 mg every 2 weeks in |
| BAVENCIO as an | (RCC) | | | combination with axitinib 5 |
| intravenous infusion | | | | mg orally twice daily. |
| over 60 minutes. | | | | |
| 200 mg/10 mL | | | | |
| (20 mg/mL) solution | | | | |
| in single-dose vial. | | | | |

In some embodiments, methods for treating cancer in a subject are provided. In some embodiments, the methods comprise administering to a subject with cancer (1) an anti-TIGIT antibody, and (2) an anti-PD-1 antibody or an anti-PD-L1 antibody; wherein the level of PD-L1 in a sample of the cancer is less than 10 as measured by Combined Positive Score (CPS) or less than 50% as measured by Total Proportion Score (TPS) or less than 50% as measured by Total Proportion Score (TPS), or less than 50% as measured by a Tumor Cell score (TC) or less than 10% as measured by Tumor-Infiltrating Immune Cell staining (IC), and wherein the anti-TIGIT antibody comprises an Fc region with enhanced effector function.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the anti-PD-1 antibody or anti-PD-L1 antibody is administered at a sub-therapeutic dose.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer is selected from small cell lung cancer, early-stage small cell lung cancer, renal cell carcinoma, urothelial cancer, triple negative breast cancer, gastric cancer, hepatocellular carcinoma, glioblastoma, ovarian cancer, head and neck squamous cell carcinoma, esophageal squamous cell carcinoma (ESCC), and non-microsatellite instability high (non-MSI high) colorectal cancer. In some embodiments, the method is the first line treatment for urothelial cancer.

In some embodiments, the methods comprise administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer comprises a mutation that reduces the efficacy of the anti-PD-1 antibody or anti-PD-L1 antibody.

In some embodiments, the methods comprise administering to a subject with cancer an anti-PD-1 antibody and an anti-PD-L1 antibody. In some embodiments, the methods comprise administering to a subject with cancer an anti-PD-1 antibody but not an anti-PD-L1 antibody. In some embodiments, the methods comprise administering to a subject with cancer an anti-PD-L1 antibody but not an anti-PD-1 antibody.

### PD-L1 Threshold Levels

In some embodiments, the cancer comprises a level of PD-L1 that is less than 10, less than 5, or less than 3, or less than 1, as measured by CPS. In some embodiments, the cancer comprises a level of PD-L1 that is between 0 and 10, or between 1 and 10, or between 3 and 10, or between 5 and 10, or between 0 and 7, or between 1 and 7, or between 3 and 7, or between 0 and 5, or between 1 and 5, or between 3 and 5, or between 0 and 3, or between 1 and 3, as measured by CPS.

In some embodiments, the cancer comprises a level of PD-L1 that is less than 50%, or less than 40%, or less than 30%, or less than 20%, or less than 10%, or less than 5%, or less than 3%, or less than 1%, as measured by TPS. In some embodiments, the cancer comprises a level of PD-L1 that is between 0% and 50%, or between 1% and 50%, or between 3% and 50%, or between 5% and 50%, or between 10% and 50%, or between 20% and 50%, or between 0% and 30%, or between 1% and 30%, or between 3% and 30%, or between 5% and 30%, or between 10% and 30%, or between 0% and 20%, or between 3% and 20%, or between 5% and 20%, as measured by TPS.

In some embodiments, the cancer comprises a level of PD-L1 that is less than 50%, or less than 40%, or less than 30%, or less than 20%, or less than 10%, or less than 5%, or less than 3%, or less than 1%, as measured by TC. In some embodiments, the cancer comprises a level of PD-L1 that is between 0% and 50%, or between 1% and 50%, or between 3% and 50%, or between 5% and 50%, or between 10% and 50%, or between 20% and 50%, or between 0% and 30%, or between 1% and 30%, or between 3% and 30%, or between 5% and 30%, or between 10% and 30%, or between 0% and 20%, or between 3% and 20%, or between 5% and 20%, as measured by TC.

In some embodiments, the cancer comprises a level of PD-L1 that is less than 10%, less than 5%, or less than 3%, or less than 1%, as measured by IC. In some embodiments, the cancer comprises a level of PD-L1 that is between 0% and 10%, or between 1% and 10%, or between 3% and 10%, or between 5% and 10%, or between 0% and 7%, or between 1% and 7%, or between 3% and 7%, or between 0% and 5%, or between 1% and 5%, or between 3% and 5%, or between 0% and 3%, or between 1% and 3%, as measured by IC.

### Dosing of Anti-TIGIT Antibody and Anti-PD-1 Antibody or Anti-PD-L1 Antibody

In some embodiments, the anti-PD-1 antibody but not the anti-PD-L1 antibody is administered at a sub-therapeutic dose. In some embodiments, the anti-PD-L1 antibody but not the anti-PD-1 antibody is administered at a sub-therapeutic dose. In some embodiments, each of the anti-PD-L1 antibody and the anti-PD-1 antibody is administered at a sub-therapeutic dose. In some embodiments, the anti-TIGIT antibody is administered at a sub-therapeutic dose.

In some embodiments, the sub-therapeutic dose of the anti-PD-1 antibody or anti-PD-L1 antibody: a) is lower than the monotherapy dose of the antibody for the cancer being treated and/or b) comprises less frequent dosing of the antibody than the frequency of monotherapy dosing for the cancer being treated. In some embodiments, the sub-therapeutic dose of the anti-TIGIT antibody a) is lower than the monotherapy dose of the anti-TIGIT antibody for the cancer being treated and/or b) comprises less frequent dosing of the anti-TIGIT antibody than the frequency of monotherapy dosing for the cancer being treated. In some embodiments, the sub-therapeutic dose of the anti-PD-1 antibody or anti-PD-L1 antibody: a) is lower than the monotherapy dose of the antibody for the cancer being treated and/or b) comprises less frequent dosing of the antibody than the frequency of monotherapy dosing for the cancer being treated; and the sub-therapeutic dose of the anti-TIGIT antibody a) is lower than the monotherapy dose of the anti-TIGIT antibody for the cancer being treated and/or b) comprises less frequent dosing of the anti-TIGIT antibody than the frequency of monotherapy dosing for the cancer being treated.

In some embodiments, the sub-therapeutic dose of the antibody includes a dose that is lower than the monotherapy dose of the antibody for the cancer being treated. In some embodiments, the sub-therapeutic dose is a dose of the antibody that is between 5% and 90%, or 5% and 80%, or 5% and 70%, or 5% and 60%, or 5% and 50%, or 5% and 40%, or 5% and 30%, or 10% and 90%, or 10% and 80%, or 10% and 70%, or 10% and 60%, or 10% and 50%, or 10% and 40%, or 10% and 30%, or 20% and 90%, or 20% and 80%, or 20% and 70%, or 20% and 60%, or 20% and 50%, or 20% and 40%, or 20% and 30%, or 30% and 90%, or 50% and 80%, or 30% and 70%, or 30% and 60%, or 30% and 50%, or 30% and 40%, or 40% and 90%, or 40% and 80%, or 40% and 70%, or 40% and 60%, or 40% and 50%, or 50% and 90%, or 50% and 80%, or 50% and 70%, or 50% and 60% of a monotherapy dose for the cancer being treated.

In some embodiments, the sub-therapeutic dose is dosing of the antibody that is less frequent than the monotherapy dosing of the antibody. In some embodiments, when monotherapy dosing is weekly, for sub-therapeutic dosing the antibody is administered every 10 days, or every 2 weeks, or every 3 weeks, or every 4 weeks, or every month, or even less frequently. In some embodiments, when monotherapy dosing is every 2 weeks, for sub-therapeutic dosing the antibody is administered every 3 weeks, or every 4 weeks, or every month, or every 5 weeks, or every 6 weeks, or even less frequently. In some embodiments, when monotherapy dosing is every 3 weeks, for sub-therapeutic dosing the antibody is administered every 4 weeks, or every month, or every 5 weeks, or every 6 weeks, or every 8 weeks, or every 2 months, or every 10 weeks, or every 12 weeks, or every 3 months, or even less frequently. In some embodiments, when monotherapy dosing is every 4 weeks, for sub-therapeutic dosing the antibody is administered every 5 weeks, or every 6 weeks, or every 8 weeks, or every 2 months, or every 10 weeks, or every 12 weeks, or every 3 months, or every 14 weeks, or every 16 weeks, or every 4 months, or even less frequently.

In some embodiments, a sub-therapeutic dose for the anti-PD-1 antibody is less than 240 mg every 2 weeks, less than 200 mg every 3 weeks, less than 350 mg every 3 weeks, less than 360 mg every 3 weeks, less than 480 mg every 4 weeks, or less than 400 mg every 6 weeks. In some embodiments, a sub-therapeutic dose for the anti-PD-1 antibody is less than 200 mg every 2 weeks, less than 150 mg every 3 weeks, less than 300 mg every 3 weeks, less than 320 mg every 3 weeks, less than 420 mg every 4 weeks, or less than 350 mg every 6 weeks. In some embodiments, a sub-therapeutic dose for the anti-PD-1 antibody is less than 150 mg every 2 weeks, less than 120 mg every 3 weeks, less than 250 mg every 3 weeks, less than 280 mg every 3 weeks, less than 360 mg every 4 weeks, or less than 300 mg every 6 weeks. In some embodiments, a sub-therapeutic dose for the anti-PD-1 antibody is less than 100 mg every 2 weeks, less than 80 mg every 3 weeks, less than 200 mg every 3 weeks, less than 240 mg every 3 weeks, less than 320 mg every 4 weeks, or less than 250 mg every 6 weeks. In some embodiments, a sub-therapeutic dose for the anti-PD-1 antibody is less than 50 mg every 2 weeks, less than 60 mg every 3 weeks, less than 150 mg every 3 weeks, less than 200 mg every 3 weeks, less than 240 mg every 4 weeks, or less than 200 mg every 6 weeks. In some embodiments, a sub-therapeutic dose for the anti-PD-1 antibody is less than 25 mg every 2 weeks, less than 20 mg every 3 weeks, less than 100 mg every 3 weeks, less than 120 mg every 3 weeks, less than 180 mg every 4 weeks, or less than 160 mg every 6 weeks.

In some embodiments, the methods comprise administering an anti-PD-1 antibody, wherein the anti-PD-1 antibody is pembrolizumab, wherein pembrolizumab is administered at a monotherapy dose or a sub-therapeutic dose that is lower than the monotherapy dose (such as within the percentages or at the reduced doses or frequencies provided herein), and wherein the monotherapy dose is 200 mg or 400 mg. In some embodiments, the methods comprise administering an anti-PD-1 antibody, wherein the anti-PD-1 antibody is nivolumab, wherein nivolumab is administered at a monotherapy dose or a sub-therapeutic dose that is lower than the monotherapy dose (such as within the percentages or at the reduced doses or frequencies provided herein), and wherein the monotherapy dose is 240 mg, 360 mg, or 480 mg. In some embodiments, the methods comprise administering an anti-PD-1 antibody, wherein the anti-PD-1 antibody is cemiplimab, wherein cemiplimab is administered at a monotherapy dose or a sub-therapeutic dose that is lower than the monotherapy dose (such as within the percentages or at the reduced doses or frequencies provided herein), and wherein the monotherapy dose is 350 mg.

In some embodiments, a sub-therapeutic dose for the anti-PD-1 antibody is less frequent than 240 mg every 2 weeks, less than 200 mg every 3 weeks, less than 350 mg every 3 weeks, less than 360 mg every 3 weeks, less than 480 mg every 4 weeks, or less than 400 mg every 6 weeks. In some embodiments, a sub-therapeutic dose for the anti-PD-1 antibody is less frequent than 240 mg every 4 weeks, less than 200 mg every 6 weeks, less than 350 mg every 6 weeks, less than 360 mg every 6 weeks, less than 480 mg every 8 weeks, or less than 400 mg every 12 weeks. In some embodiments, a sub-therapeutic dose for the anti-PD-1 antibody is less frequent than 240 mg every 6 weeks, less than 200 mg every 9 weeks, less than 350 mg every 9 weeks, less than 360 mg every 9 weeks, less than 480 mg every 12 weeks, or less than 400 mg every 18 weeks. In some embodiments, a sub-therapeutic dose for the anti-PD-1 antibody is less frequent than 240 mg every 8 weeks, less than 200 mg every 12 weeks, less than 350 mg every 12 weeks, less than 360 mg every 12 weeks, less than 480 mg every 16 weeks, or less than 400 mg every 24 weeks. In some embodiments, a sub-therapeutic dose for the anti-PD-1 antibody is less frequent than 240 mg every 10 weeks, less than 200 mg every 15 weeks, less than 350 mg every 15 weeks, less than 360 mg every 15 weeks, less than 480 mg every 20 weeks, or less than 400 mg every 30 weeks.

In some embodiments, the methods comprise administering an anti-PD-1 antibody, wherein the anti-PD-1 antibody is pembrolizumab, wherein pembrolizumab is administered at a monotherapy dose or a sub-therapeutic dose that is lower than the monotherapy dose (such as within the percentages or at the reduced doses or frequencies provided herein), and wherein the frequency of monotherapy dosing is every 3 weeks or every 6 weeks. In some embodiments, the methods comprise administering an anti-PD-1 antibody, wherein the anti-PD-1 antibody is pembrolizumab, wherein pembrolizumab is administered at a monotherapy dose or a sub-therapeutic dose that is lower than the monotherapy dose (such as within the percentages or at the reduced doses or frequencies provided herein), and wherein the monotherapy dose is 200 mg every 3 weeks or 400 mg every 6 weeks. In some embodiments, the methods comprise administering an anti-PD-1 antibody, wherein the anti-PD-1 antibody is nivolumab, wherein nivolumab is administered at a monotherapy dose or a sub-therapeutic dose that is lower than the monotherapy dose (such as within the percentages or at the reduced doses or frequencies provided herein), and wherein the frequency of monotherapy dosing is every 2 weeks or every 3 weeks or every 4 weeks. In some embodiments, the methods comprise administering an anti-PD-1 antibody, wherein the anti-PD-1 antibody is nivolumab, wherein nivolumab is administered at a monotherapy dose or a sub-therapeutic dose that is lower than the monotherapy dose (such as within the percentages or at the reduced doses or frequencies provided herein), and wherein the monotherapy dose is 240 mg every 2 weeks, 360 mg every 3 weeks, or 480 mg every 4 weeks. In some embodiments, the methods comprise administering an anti-PD-1 antibody, wherein the anti-PD-1 antibody is cemiplimab, wherein cemiplimab is administered at a monotherapy dose or a sub-therapeutic dose that is lower than the monotherapy dose (such as within the percentages or at the reduced doses or frequencies provided herein), and wherein the frequency of monotherapy dosing is every 3 weeks.

In some embodiments, a sub-therapeutic dose for the anti-PD-L1 antibody is less than 800 mg every 2 weeks, less than 840 mg every 2 weeks, less than 1,200 mg every 3 weeks, less than 1,500 mg every 3 weeks, or less than 1,680 mg every 4 weeks. In some embodiments, a sub-therapeutic dose for the anti-PD-L1 antibody is less than 600 mg every 2 weeks, less than 620 mg every 2 weeks, less than 800 mg every 3 weeks, less than 1,000 mg every 3 weeks, or less than 1,240 mg every 4 weeks. In some embodiments, a sub-therapeutic dose for the anti-PD-L1 antibody is less than 400 mg every 2 weeks, less than 410 mg every 2 weeks, less than 400 mg every 3 weeks, less than 500 mg every 3 weeks, or less than 820 mg every 4 weeks. In some embodiments, a sub-therapeutic dose for the anti-PD-L1 antibody is less than 200 mg every 2 weeks, less than 200 mg every 3 weeks, less than 250 mg every 3 weeks, or less than 410 mg every 4 weeks.

In some embodiments, the methods comprise administering an anti-PD-L1 antibody, wherein the anti-PD-L1 antibody is avelumab, wherein avelumab is administered at a monotherapy dose or a sub-therapeutic dose that is lower than the monotherapy dose (such as within the percentages or at the reduced doses or frequencies provided herein), and wherein the monotherapy dose is 800 mg. In some embodiments, the methods comprise administering an anti-PD-L1 antibody, wherein the anti-PD-L1 antibody is durvalumab, wherein durvalumab is administered at a monotherapy dose or a sub-therapeutic dose that is lower than the monotherapy dose (such as within the percentages or at the reduced doses or frequencies provided herein), and wherein the monotherapy dose is 10 mg/kg or 1,500 mg. 20.

In some embodiments, a sub-therapeutic dose for the anti-PD-L1 antibody is less frequent than 800 mg every 2 weeks, less than 840 mg every 2 weeks, less than 1,200 mg every 3 weeks, less than 1,500 mg every 3 weeks, or less than 1,680 mg every 4 weeks. In some embodiments, a sub-therapeutic dose for the anti-PD-L1 antibody is less frequent than 800 mg every 4 weeks, less than 840 mg every 4 weeks, less than 1,200 mg every 6 weeks, less than 1,500 mg every 6 weeks, or less than 1,680 mg every 8 weeks. In some embodiments, a sub-therapeutic dose for the anti-PD-L1 antibody is less frequent than 800 mg every 6 weeks, less than 840 mg every 6 weeks, less than 1,200 mg every 9 weeks, less than 1,500 mg every 9 weeks, or less than 1,680 mg every 12 weeks. In some embodiments, a sub-therapeutic dose for the anti-PD-L1 antibody is less frequent than 800 mg every 8 weeks, less than 840 mg every 8 weeks, less than 1,200 mg every 12 weeks, less than 1,500 mg every 12 weeks, or less than 1,680 mg every 16 weeks. In some embodiments, a sub-therapeutic dose for the anti-PD-L1 antibody is less frequent than 800 mg every 10 weeks, less than 840 mg every 10 weeks, less than 1,200 mg every 15 weeks, less than 1,500 mg every 15 weeks, or less than 1,680 mg every 20 weeks.

In some embodiments, the methods comprise administering an anti-PD-L1 antibody, wherein the anti-PD-L1 antibody is atezolizumab, wherein atezolizumab is administered at a monotherapy dose or a sub-therapeutic dose that is lower than the monotherapy dose (such as within the percentages or at the reduced doses or frequencies provided herein), and wherein the monotherapy dose is 840 mg, 1,200 mg, or 1,680 mg. In some embodiments, the methods comprise administering an anti-PD-L1 antibody, wherein the anti-PD-L1 antibody is avelumab, wherein avelumab is administered at a monotherapy dose or a sub-therapeutic dose that is lower than the monotherapy dose (such as within the percentages or at the reduced doses or frequencies provided herein), wherein the frequency of monotherapy dosing is every 2 weeks. In some embodiments, the methods comprise administering an anti-PD-L1 antibody, wherein the anti-PD-L1 antibody is durvalumab, wherein durvalumab is administered at a monotherapy dose or a sub-therapeutic dose that is lower than the monotherapy dose (such as within the percentages or at the reduced doses or frequencies provided herein), wherein the frequency of monotherapy dosing is every 2 weeks or every 4 weeks. In some embodiments, the methods comprise administering an anti-PD-L1 antibody, wherein the anti-PD-L1 antibody is durvalumab, wherein durvalumab is administered at a monotherapy dose or a sub-therapeutic dose that is lower than the monotherapy dose (such as within the percentages or at the reduced doses or frequencies provided herein), and wherein the monotherapy dose is 10 mg/kg mg every 2 weeks or 1,500 mg every 4 weeks. In some embodiments, the methods comprise administering an anti-PD-L1 antibody, wherein the anti-PD-L1 antibody is atezolizumab, wherein atezolizumab is administered at a monotherapy dose or a sub-therapeutic dose that is lower than the monotherapy dose (such as within the percentages or at the reduced doses or frequencies provided herein), wherein the frequency of monotherapy dosing is every 2 weeks, every 3 weeks, or every 4 weeks. In some embodiments, the methods comprise administering an anti-PD-L1 antibody, wherein the anti-PD-L1 antibody is atezolizumab, wherein atezolizumab is administered at a monotherapy dose or a sub-therapeutic dose that is lower than the monotherapy dose (such as within the percentages or at the reduced doses or frequencies provided herein), and wherein the monotherapy dose is 840 mg every 2 weeks, 1,200 mg every 3 weeks, or 1,680 mg every 4 weeks.

In some embodiments, a sub-therapeutic dose of the anti-TIGIT antibody includes a dose that is lower than the monotherapy dose of the anti-TIGIT antibody for the cancer being treated. In some embodiments, the sub-therapeutic dose is a dose of the anti-TIGIT antibody that is between 5% and 90%, or 5% and 80%, or 5% and 70%, or 5% and 60%, or 5% and 50%, or 5% and 40%, or 5% and 30%, or 10% and 90%, or 10% and 80%, or 10% and 70%, or 10% and 60%, or 10% and 50%, or 10% and 40%, or 10% and 30%, or 20% and 90%, or 20% and 80%, or 20% and 70%, or 20% and 60%, or 20% and 50%, or 20% and 40%, or 20% and 30%, or 30% and 90%, or 50% and 80%, or 30% and 70%, or 30% and 60%, or 30% and 50%, or 30% and 40%, or 40% and 90%, or 40% and 80%, or 40% and 70%, or 40% and 60%, or 40% and 50%, or 50% and 90%, or 50% and 80%, or 50% and 70%, or 50% and 60% of the monotherapy dose for the cancer being treated.

The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a lower dose, which is then increased to a higher dose tolerable to the patient. In some embodiments, a patient initially is given a higher dose, which is then decreased to a lower dose.

### Exemplary Indications

In some embodiments, the cancer is bladder cancer, breast cancer, triple negative breast cancer, uterine cancer, cervical cancer, ovarian cancer, prostate cancer, testicular cancer, esophageal cancer, esophageal squamous cell carcinoma (ESCC), gastrointestinal cancer, gastric cancer, pancreatic cancer, colorectal cancer, non-microsatellite instability high (non-MSI high) colorectal cancer, colon cancer, kidney cancer, renal cell carcinoma, clear cell renal carcinoma, head and neck cancer, glioblastoma, lung cancer, small cell lung cancer, early-stage small cell lung cancer, lung adenocarcinoma, stomach cancer, germ cell cancer, bone cancer, liver cancer, hepatocellular carcinoma, thyroid cancer, skin cancer, melanoma, neoplasm of the central nervous system, mesothelioma, lymphoma, leukemia, chronic lymphocytic leukemia, diffuse large B cell lymphoma, follicular lymphoma, Hodgkin lymphoma, myeloma, or sarcoma. In some embodiments, the cancer is selected from gastric cancer, testicular cancer, pancreatic cancer, lung adenocarcinoma, bladder cancer, urothelial cancer, head and neck cancer, head and neck squamous cell carcinoma, prostate cancer, mesothelioma, and clear cell renal carcinoma. In some embodiments, the cancer is a lymphoma or a leukemia, including but not limited to acute myeloid, chronic myeloid, acute lymphocytic or chronic lymphocytic leukemia, diffuse large B-cell lymphoma, follicular lymphoma, mantle cell lymphoma, small lymphocytic lymphoma, primary mediastinal large B-cell lymphoma, splenic marginal zone B-cell lymphoma, or extranodal marginal zone B-cell lymphoma. In some embodiments, the cancer is colorectal cancer, colon cancer, kidney cancer, or clear cell renal carcinoma. In some embodiments, the cancer is a metastatic cancer.

In some embodiments, the cancer is selected from small cell lung cancer, early-stage small cell lung cancer, renal cell carcinoma, urothelial cancer, triple negative breast cancer, gastric cancer, hepatocellular carcinoma, glioblastoma, ovarian cancer, head and neck squamous cell carcinoma, esophageal squamous cell carcinoma (ESCC), and non-microsatellite instability high (non-MSI high) colorectal cancer.

In some embodiments, the cancer is non-small cell lung cancer.

In some embodiments, the cancer is one with high tumor mutation burden as such cancers often have more antigen to drive T cell responses. Thus, in some embodiments, the cancer is a high mutational burden cancer such as lung, melanoma, bladder, or gastric cancer. In some embodiments, the cancer has microsatellite instability.

In some embodiments,
a) the cancer is non-small cell lung cancer, and the TPS is <1%;
b) the cancer is head and neck squamous cell cancer (HNSCC), and the CPS is <1;
c) the cancer is urothelial carcinoma, and the CPS is <10;
d) the cancer is gastric cancer, and the CPS is <1;
e) the cancer is esophageal cancer, and the CPS is <10;
f) the cancer is cervical cancer, and the CPS is <1; or
g) the cancer is triple negative breast cancer, and the CPS is <10.

In some embodiments,
a) the cancer is urothelial carcinoma, and the IC is <5%;
b) the cancer is triple-negative breast cancer, and the IC is <1%; or
c) the cancer is non-small cell lung cancer, and the IC is <10%.

In some embodiments, the cancer is non-small cell lung cancer, and the TPS is <50%.

In some embodiments, the methods are first line treatments of urothelial cancer.

In some embodiments, the cancer comprises a mutation that reduces the efficacy of the anti-PD-1 antibody and/or anti-PD-L1 antibody. In some embodiments, the cancer comprises a mutation that reduces the efficacy of each of the anti-PD-1 antibody and the anti-PD-L1 antibody. In some embodiments, the cancer comprises a mutation that reduces the efficacy of the anti-PD-1 antibody but not the anti-PD-L1 antibody. In some embodiments, the cancer comprises a mutation that reduces the efficacy of the anti-PD-L1 antibody but not the anti-PD-L1 antibody. In some embodiments, the cancer comprises a mutation that reduces the efficacy of the anti-TIGIT antibody.

In some embodiments, the cancer comprises a mutation in the EGFR gene and/or a mutation in the ALK gene and/or a mutation in the ROS1 gene. In some embodiments, the cancer comprises a mutation in the EGFR gene and/or a mutation in the ALK gene. In some embodiments, the cancer comprises a mutation in the EGFR gene and a mutation in the ALK gene but not a mutation in the ROS1 gene. In some embodiments, the cancer comprises a mutation in the EGFR gene and a mutation in the ROS1 gene but not a mutation in the ALK gene. In some embodiments, the cancer comprises a mutation in the ALK gene and a mutation in the ROS1 gene but not a mutation in the EGFR gene. In some embodiments, the cancer comprises a mutation in the EGFR gene but not a mutation in the ALK gene or a mutation in the ROS1 gene. In some embodiments, the cancer comprises a mutation in the ALK gene but not a mutation in the EGFR gene or a mutation in the ROS1 gene. In some embodiments, the cancer comprises a mutation in the ROS1 gene but not a mutation in the ALK gene or a mutation in the EGFR gene.

### Further Exemplary Embodiments

In some embodiments, the anti-TIGIT antibody comprises an Fc with enhanced binding to at least one of FcγRIIIa, FcγRIIa, and FcγRI. In some embodiments, the anti-TIGIT antibody comprises an Fc with enhanced binding to each of FcγRIIIa, FcγRIIa, and FcγRI. In some embodiments, the anti-TIGIT antibody comprises an Fc with enhanced binding to at least FcγRIIIa and FcγRIIa. In some embodiments, the anti-TIGIT antibody comprises an Fc with enhanced binding to at least FcγRIIIa and FcγRI. In some embodiments, the anti-TIGIT antibody comprises an Fc with enhanced binding to at least FcγRIIa and FcγRI. In some embodiments, the anti-TIGIT antibody comprises an Fc with enhanced binding to at least FcγRIIIa. In some embodiments, the anti-TIGIT antibody comprises an Fc with enhanced binding to at least FcγRIIa. In some embodiments, the anti-TIGIT antibody comprises an Fc with enhanced binding to at least FcγRI.

In some embodiments, the Fc of the anti-TIGIT antibody has reduced binding to one or more inhibitory FcγRs.

In some embodiments, the Fc of the anti-TIGIT antibody has reduced binding to FcγRIIb.

In some embodiments, the anti-TIGIT antibody comprises substitutions S293D, A330L, and I332E in the heavy chain constant region.

In some embodiments, the anti-TIGIT antibody is nonfucosylated. In some embodiments, the anti-TIGIT antibody is comprised in a composition of anti-TIGIT antibodies, wherein at least 90%, at least 91%, at least 92%, at least 93%, at least 94%, at least 95%, at least 96%, at least 97%, at least 98%, or at least 99% of the anti-TIGIT antibodies in the composition are nonfucosylated.

In some embodiments, the Fc of the anti-TIGIT antibody comprises an Fc with enhanced ADCC and/or ADCP activity relative to a corresponding wild-type Fc of the same isotype.

In some embodiments, the anti-TIGIT antibody comprises:
a) a heavy chain CDR1 comprising an amino acid sequence selected from SEQ ID NOs: 7-9;
b) a heavy chain CDR2 comprising an amino acid sequence selected from SEQ ID NOs: 10-13;
c) a heavy chain CDR3 comprising an amino acid sequence selected from SEQ ID NOs: 14-16;
d) a light chain CDR1 comprising the amino acid sequence of SEQ ID NO: 17;
e) a light chain CDR2 comprising the amino acid sequence of SEQ ID NO: 18; and
f) a light chain CDR3 comprising the amino acid sequence of SEQ ID NO: 19.

In some embodiments, the anti-TIGIT antibody comprises a heavy chain CDR1, CDR2, and CDR3 and a light chain CDR1, CDR, and CDR3 comprising the sequences of:
a) SEQ ID NOs: 7, 10, 14, 17, 18, and 19, respectively; or
b) SEQ ID NOs: 8, 11, 14, 17, 18, and 19, respectively; or
c) SEQ ID NOs: 9, 12, 15, 17, 18, and 19, respectively; or
d) SEQ ID NOs: 8, 13, 16, 17, 18, and 19, respectively; or
e) SEQ ID NOs: 8, 12, 16, 17, 18, and 19, respectively.

In some embodiments, the anti-TIGIT antibody comprises a heavy chain variable region comprising an amino acid sequence selected from SEQ ID NOs: 1-5 and a light chain variable region comprising the amino acid sequence of SEQ ID NO: 6.

In some embodiments, the anti-TIGIT antibody comprises a heavy chain comprising an amino acid sequence selected from SEQ ID NOs: 20-24 and a light chain comprising the amino acid sequence of SEQ ID NO: 25.

In some embodiments, the methods comprise administering an anti-PD-1 antibody or multiple anti-PD-1 antibodies.

In some embodiments, the anti-PD-1 antibody is selected from or each of the multiple anti-PD-1 antibodies is independently selected from pembrolizumab, nivolumab, CT-011, BGB-A317, cemiplimab, sintilimab, tislelizumab, TSR-042, PDR001, or toripalimab.

In some embodiments, the methods comprise administering an anti-PD-1 antibody, wherein the anti-PD-1 antibody is pembrolizumab or nivolumab. In some embodiments, the methods comprise administering an anti-PD-1 antibody, wherein the anti-PD-1 antibody is cemiplimab. In some embodiments, the methods comprise administering an anti-PD-L1 antibody, wherein the anti-PD-L1 antibody is atezolizumab.

In some embodiments, the methods comprise administering an anti-PD-L1 antibody or multiple anti-PD-L1 antibodies.

In some embodiments, the anti-PD-L1 antibody is selected from or each of the multiple anti-PD-L1 antibodies is independently selected from durvalumab, BMS-936559, atezolizumab, or avelumab.

In some embodiments, the methods comprise administering an anti-PD-1 antibody, wherein the anti-PD-1 antibody is pembrolizumab or nivolumab; or wherein the method comprises administering an anti-PD-L1 antibody, wherein the anti-PD-L1 antibody is atezolizumab

In various embodiments, the anti-TIGIT antibody depletes T regulatory (Treg) cells, activates antigen presenting cells (APCs), enhances CD8 T cell responses, upregulates co-stimulatory receptors, and/or promotes release of immune activating cytokines (such as CXCL10 and/or IFNγ). In some embodiments, the anti-TIGIT antibody promotes release of immune activating cytokines to a greater extent than immune suppressive cytokines (such as IL10 and/or MDC).

The anti-TIGIT antibody, the anti-PD-1 antibody, and/or the anti-PD-L1 antibody may be administered concurrently or sequentially. For sequential administration, at least a first dose of one of the anti-TIGIT antibody, the anti-PD-1 antibody, and the anti-PD-L1 antibody may be administered before at least a first dose of another of the anti-TIGIT antibody, the anti-PD-1 antibody, and the anti-PD-L1 antibody. For concurrent administration, in some embodiments, at least a first dose of one of the anti-TIGIT antibody, the anti-PD-1 antibody, and the anti-PD-L1 antibody and at least a first dose of another of the anti-TIGIT antibody, the anti-PD-1 antibody, and the anti-PD-L1 antibody may be administered as separate pharmaceutical compositions or in the same pharmaceutical composition.

The route of administration of a pharmaceutical composition can be oral, intraperitoneal, transdermal, subcutaneous, intravenous, intramuscular, inhalational, topical, intralesional, rectal, intrabronchial, nasal, transmucosal, intestinal, ocular or otic delivery, or any other methods known in the art. In some embodiments, one or more therapeutic agents are administered orally, intravenously, or intraperitoneally.

Co-administered therapeutic agents, such as any of the anti-TIGIT antibody, the anti-PD-1 antibody, and/or the anti-PD-L1 antibody, can be administered together or separately, simultaneously or at different times. When administered, the therapeutic agents independently can be administered once, twice, three, four times daily or more or less often, as needed. In some embodiments, the administered therapeutic agents are administered once daily. In some embodiments, the administered therapeutic agents are administered at the same time or times, for instance as an admixture. In some embodiments, one or more of the therapeutic agents is administered in a sustained-release formulation.

In some embodiments, any of the combination therapies provided herein is administered to the subject over an extended period of time, e.g., for at least 30, 40, 50, 60, 70, 80, 90, 100, 150, 200, 250, 300, 350 days or longer.

### Exemplary Efficacy Outcomes

In some embodiments, the enhanced activity observed with at least some of the combination therapies described herein have certain benefits as compared to corresponding monotherapy treatment. For example, in some embodiments, the combination therapies have toxicity profiles comparable to that of any of the component antibodies when administered as a monotherapy. In some embodiments, administration of the combination therapies provides a longer duration of response as compared to that of any of the component antibodies when administered as a monotherapy. In some embodiments, administration of the combination therapies results in longer progression-free survival as compared to that of any of the component antibodies when administered as a monotherapy. In some embodiments, administration of the combination therapies can be used to treat recurrent cancer that recurs following monotherapy treatment with any of the combination therapies' component antibodies.

### VI. Compositions and Kits

In another aspect, compositions and kits for use in treating or preventing a cancer in a subject are provided.

### Pharmaceutical Compositions

In some embodiments, pharmaceutical compositions for use in the present methods are provided. In some embodiments, at least one of (1) an anti-TIGIT antibody and (2) an anti-PD-1 antibody and/or an anti-PD-L1 antibody is administered in a first pharmaceutical composition and at least another of (1) the anti-TIGIT antibody and (2) the anti-PD-1 antibody and/or the anti-PD-L1 antibody is administered in a second pharmaceutical composition. In some embodiments, (1) the anti-TIGIT antibody and (2) the anti-PD-1 antibody and/or the anti-PD-L1 antibody are administered in a single pharmaceutical composition.

Guidance for preparing formulations for use in the present invention is found in, for example, Remington: The Science and Practice of Pharmacy, 21^{st} Ed., 2006, supra; Martindale: The Complete Drug Reference, Sweetman, 2005, London: Pharmaceutical Press; Niazi, Handbook of Pharmaceutical Manufacturing Formulations, 2004, CRC Press; and Gibson, Pharmaceutical Preformulation and Formulation: A Practical Guide from Candidate Drug Selection to Commercial Dosage Form, 2001, Interpharm Press, which are hereby incorporated herein by reference. The pharmaceutical compositions described herein can be manufactured in a manner that is known to those of skill in the art, e.g., by means of conventional mixing, dissolving, granulating, dragee-making, emulsifying, encapsulating, entrapping or lyophilizing processes. The following methods and excipients are merely exemplary and are in no way limiting.

In some embodiments, one or more therapeutic agents are prepared for delivery in a sustained-release, controlled release, extended-release, timed-release or delayed-release formulation, for example, in semi-permeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

For oral administration, a therapeutic agent can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

A therapeutic agent can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

A therapeutic agent can be administered systemically by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplary transdermal delivery formulations include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. In some embodiments, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, PA. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

Dosages and desired concentration of pharmaceutical compositions of the disclosure may vary depending on the particular use envisioned. The determination of the appropriate dosage or route of administration is well within the skill of one in the art. Suitable dosages are also described herein.

### Kits

In some embodiments, kits for use in treating a subject having a cancer are provided. In some embodiments, the kit comprises:
an anti-TIGIT antibody, as provided herein; and
an anti-PD-1 antibody and/or an anti-PD-L1 antibody, as provided herein.

In some embodiments, the kits can further comprise instructional materials containing directions (i.e., protocols) for the practice of the methods of this invention (e.g., instructions for using the kit for treating a cancer). While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### VII. Examples

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Tumor Microenvironments in Different Syngeneic Models

### 1.1 Materials and Methods

Renca, CT26, and MC38 tumor cells were implanted in Balb/c or C57BL/6 mice subcutaneously and allowed to grow to 100 mm³. The tumors were excised from the animals, and a single cell suspension was created using an enzymatic dissociation kit per the manufacturer's (Millipore) instruction. The tumors were stained with antibodies to discern B cells (CD19+), CD4+ T cells (CD3+CD8−CD4+FoxP3−), NK cells (CD3−NKp46+), mMDSCs (CD11b+Ly6G−Ly6C+), DCs (CD11c+MHCII+), gMDSCs (CD11b+Ly6G+Ly6C−), macrophages (CD11b+F4/80+Ly6c−Ly6G−), regulatory T cells (CD3+CD8−CD4+FoxP3+CD25+CD127−), CD8+ T cells (CD3+CD4−CD8+), and activated CD8+ T cells (CD3+CD4−CD8+Eomes+PD-1+Ki67+), and were analyzed using an Attuen flow cytometer.

### 1.2 Results

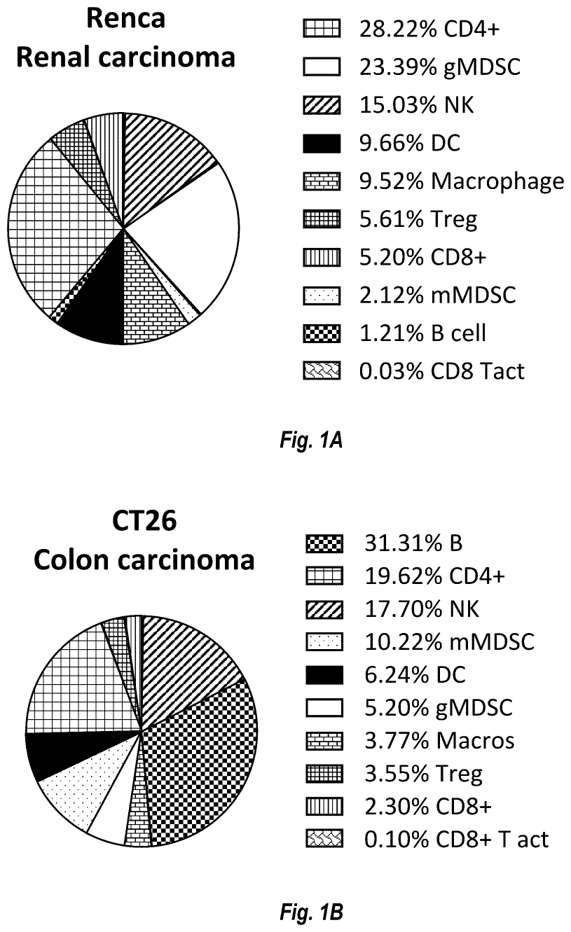

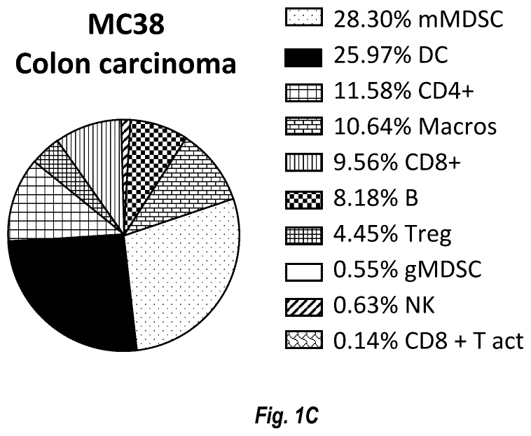

The percentage of each cell type in the tumors was determined and plotted (FIGS. 1A-1C). Each tumor, at the 100 mm³ baseline before start of treatment, showed distinct and variable levels of each immune cell subset in the tumor microenvironment. MC38, for example, showed increased enrichment with innate immune cells versus T cells (FIG. 1C). While these results are the baseline immune microenvironments in these tumor types when analyzed at the inventors' facility, it is possible the results would be different when these tumor types are evaluated at other facilities (Mosely, 2017, Cancer Immunology Res. 5(1): 29-41).

### Example 2: Tumor PD-1 and PD-L1 Expression Levels

### 1.1 Materials and Methods

Bulk tumor RNAseq was performed on Renca, CT26, and MC38 tumors of various sizes (100 mm³, 300 mm³, and 1,000 mm³). To investigate the baseline immune checkpoint characteristics of the various syngeneic tumor models that could underlie their responsiveness to different therapies, the transcriptional levels of various immune checkpoint molecules were analyzed.

### 1.2 Results

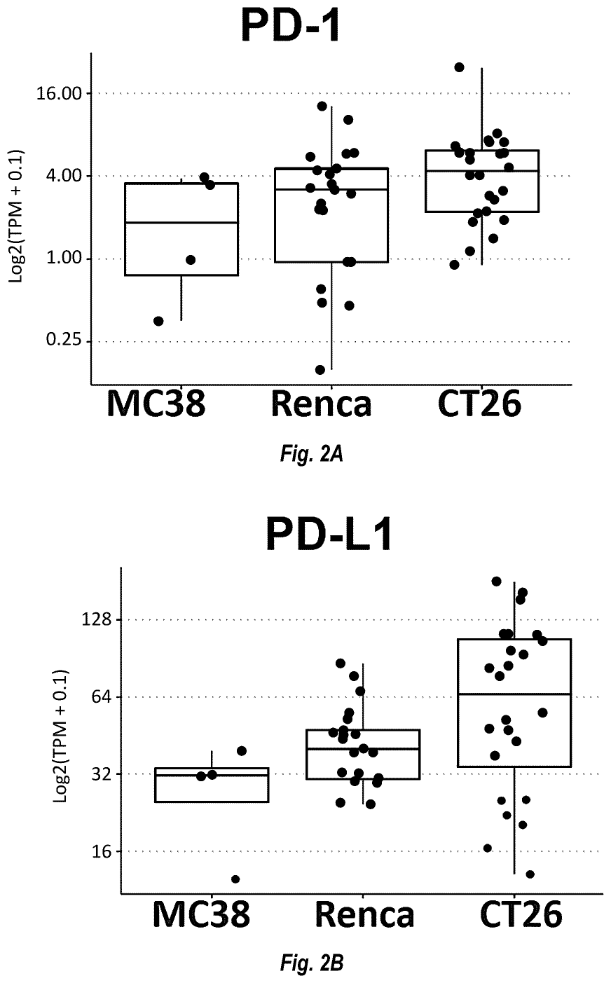

This analysis revealed disparate levels of PD-1 and PD-L1 expression between the different tumor models (FIGS. 2A-2B), with MC38 tumors displaying the lowest average levels of both molecules, followed by Renca tumors. CT26 tumors exhibited the highest levels of both molecules.

### Example 3: Responsiveness of MC38 Tumors to anti-TIGIT and anti-PD-1 Antibodies

### 1.1 Materials and Methods

Responsiveness of MC38 tumors to the combination of various anti-TIGIT antibodies with different Fc backbones and an anti-PD-1 antibody was tested by implanting C57BL/6 mice with the MC38 syngeneic tumor cell line. Tumors were implanted subcutaneously, and when they reached 100 mm³, animals were treated with 0.1 mg/kg of an SEA-TGT mIgG2a antibody (i.e., the SEA-TGT antibody reformatted as a nonfucosylated mouse IgG2a that corresponds to a nonfucosylated human IgG1 backbone), a wild type mIgG2a anti-TIGIT antibody, an Fc-null anti-TIGIT LALA mIgG2a antibody, an anti-mouse PD-1 antibody, or combinations of both agents (e.g., SEA-TGT and anti-mouse PD-1), three doses at 3-day intervals (q3dx3). Each of the different anti-TIGIT antibodies had the same variable domain and differed just with respect to the Fc backbone and the associated level of enhanced effector function (nonfucosylated >wild-type >LALA Fc-null). Tumor size was measured and growth was plotted over time.

### 1.2 Results

Animals treated with the sub-optimal dose (0.1 mg/kg) or lower doses of either agent alone only demonstrated minimal responsiveness and tumor growth delay (FIG. 3) (not shown for TIGITs alone). Addition of the Fc-null anti-TIGIT LALA antibody to the PD-1 treatment did not enhance anti-tumor activity. Addition of the standard anti-TIGIT on a mIgG2a backbone (equivalent FcγR engagement to that of a IgG1 human backbone) to the PD-1 treatment substantially increased the extent to which tumor growth was delayed and increased the cure rate two-fold (FIG. 3).

However, when this Fc interaction was further enhanced with the SEA backbone of the nonfucosylated anti-TIGIT antibody (SEA-TGT mIgG2a), the anti-tumor activity was enhanced even further and was coupled with a further two-fold increase in complete responses (FIG. 3). These data demonstrate that Fc engagement by an anti-TIGIT antibody helps drive synergy between anti-TIGIT treatment and anti-PD-1 blockade. Also, Notably, this synergistic activity with SEA-TGT mIgG2a was seen in the MC38 model which had the lowest levels of both PD-1 and PD-L1 expression (FIG. 2).

### Example 4: Responsiveness of CT26 and Renca Tumors to anti-TIGIT and anti-PD-1 Antibodies

### 1.1 Materials and Methods

Responsiveness to the combination of SEA-TGT mIgG2a (see Example 3 for additional details) and an anti-PD-1 antibody was further tested in CT26 and Renca models. Balb/c mice were implanted with CT26 or Renca syngeneic tumor cell lines. Tumors were implanted subcutaneously and when they reached 100 mm³, animals were treated with sub-optimal concentrations of (0.1 mg/kg) of SEA-TGT mIgG2a, anti-mouse PD-1, or combinations of both agents, q3dx3. Tumor size was measured and growth was plotted over time.

### 1.2 Results

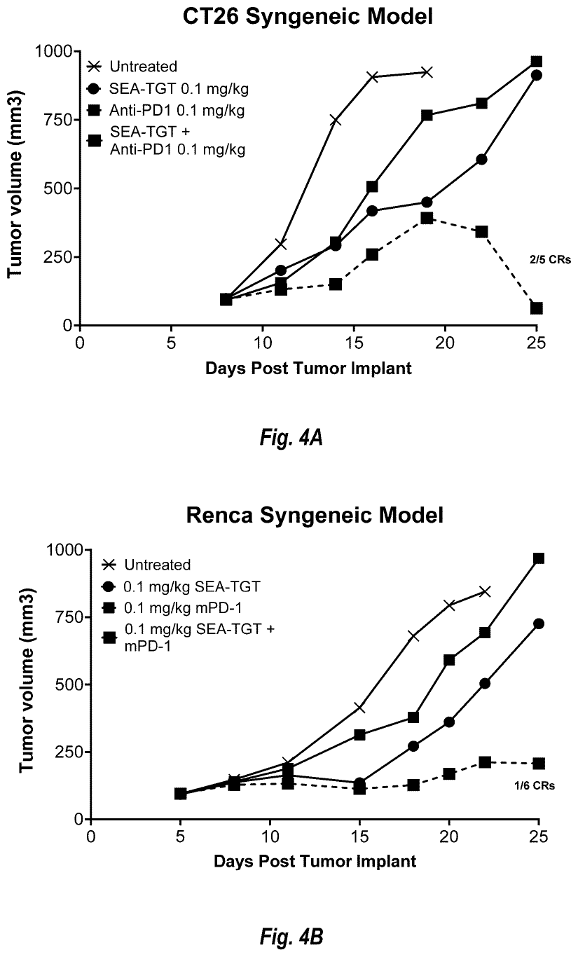

Animals treated with the sub-optimal dose of 0.1 mg/kg of SEA-TGT alone demonstrated minimal responsiveness and tumor growth delay in both the CT26 model (FIG. 4A) and the Renca model (FIG. 4B); similarly, minimal anti-tumor activity was seen with low dose anti-PD-1 treatment. However, addition of the two sub-optimal doses of SEA-TGT mIgG2a and an anti-PD-1 antibody greatly enhanced the anti-tumor activity. Increases in both tumor growth delay and complete responses to the combination treatment were seen in both the CT26 model (FIG. 4A) and the Renca model (FIG. 4B). The extent of the combinatorial activity was greater in the CT26 model (FIG. 4A) as compared with the Renca model (FIG. 4B), with the MC38 model (FIG. 3) still demonstrating the greatest sensitivity to the combination.

### 1.3 Summary of Examples 2-4

In light of the different levels of PD-L1 expression in these models (FIGS. 2A-2B), the data from Examples 2-4 demonstrate that the synergistic effects of SEA-TGT mIgG2a and an anti-mouse PD-1 antibody are surprisingly not correlated with underlying PD-L1 expression levels. The MC38 model, with the lowest PD-L1 expression levels, showed the greatest effects when treated with the combination of SEA-TGT mIgG2a and an anti-PD-1 antibody (FIG. 3).

The finding that an anti-TIGIT antibody comprising an Fc region with an enhanced effector function (e.g., a nonfucosylated antibody such as SEA-TGT) was able to exhibit synergistic effects with an anti-PD-1 antibody, even in tumor models expressing lower levels of PD-L1, was surprising. This is the case because studies with anti-TIGIT antibodies with other Fc backbones (e.g., wild-type or IgG1-effector null) have found that the combination is dependent upon relatively high levels of PD-L1 expression. As such, conventionally, clinical trials conducted with such anti-TIGIT antibodies were often designed to test the combination only in patients expressing PD-L1 above certain threshold limits.

### Example 5: Treatment of MC38, CT26, and Renca Tumors with Single Agents

### 1.1 Materials and Methods

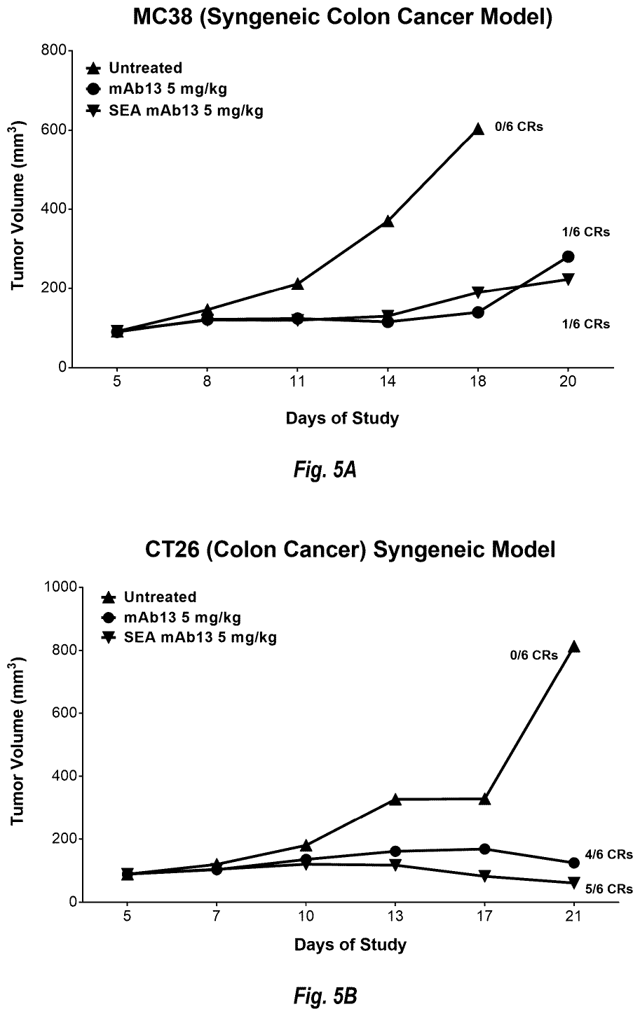

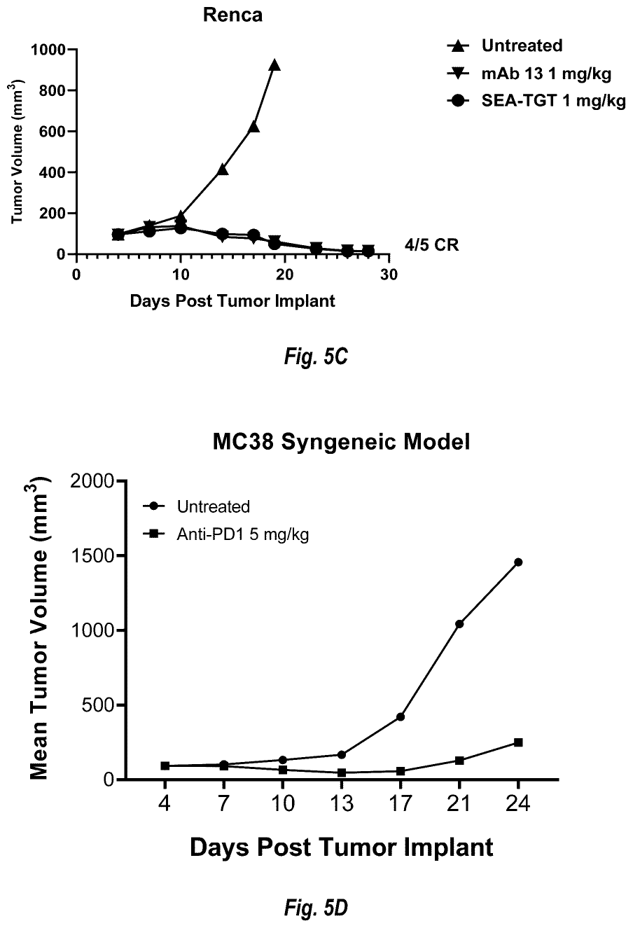

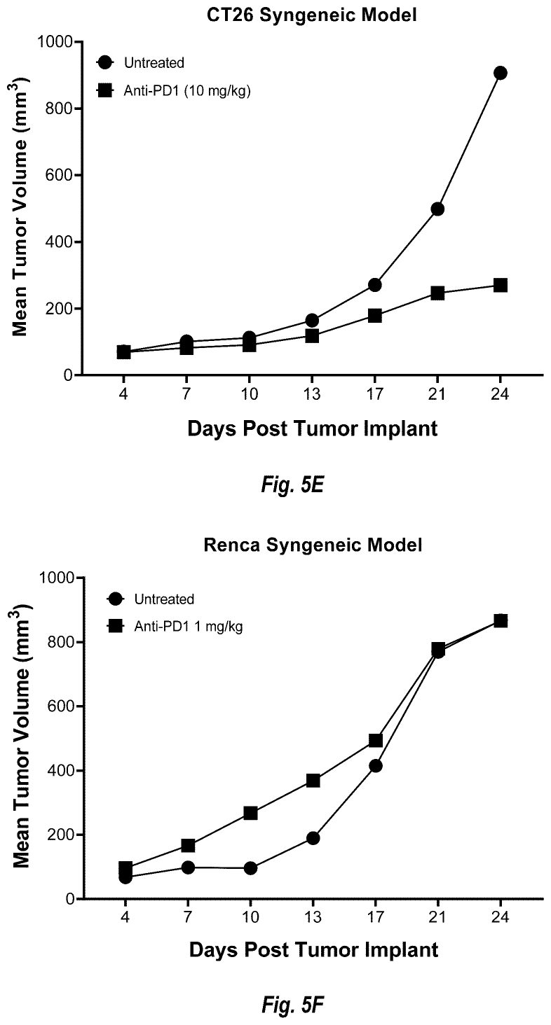

Responsiveness of MC38, CT26, and Renca tumors to anti-TIGIT antibodies with different Fc backbones was tested by implanting C57BL/6 or Balb/c mice with the indicated syngeneic tumor cell line (FIGS. 5A-5F). Tumors were implanted subcutaneously and when they reached 100 mm³, the animals were treated with the indicated doses (FIGS. 5A-5F) of a wild type fucosylated mIgG2a version of SEA-TGT (mAb13) or SEA-TGT mIgG2a (the SEA-TGT antibody reformatted as a nonfucosylated mouse IgG2a that corresponds to a nonfucosylated human IgG1 backbone), q3dx3. Tumor size was measured and growth was plotted over time. Complete tumor ablation in animals in each group was noted as complete responses (CR). Independent of backbone effector function, higher doses of clone 13 or SEA-TGT mIgG2a induced antitumor responses, but were not always able to drive the same curative responses as that seen when combined with anti-PD-1.

### 1.2 Results

In the MC38 model, even at a dose of 5 mg/kg of the anti-TIGIT treatment alone, a complete-response curative rate was only 1/6 (FIG. 5A), opposed to 4/5 when only 0.1 mg/kg of SEA-TGT mIgG2a was combined with a dose of 0.1 mg/kg of an anti-PD-1 antibody (FIG. 3). Thus, in the MC38 model, the complete response rate for the combination therapy was better than that achieved even with a 50-fold increase in the dose level of SEA-TGT mIgG2a as a monotherapy.

In the CT26 model, a 5 mg/kg dose of SEA-TGT mIgG2a drove enhanced levels of curative responses as compared to those achieved in the MC38 model (FIG. 5B), but even then a similar level of curative responses was seen with a 0.1 mg/kg dose when given with an anti-PD-1 antibody (FIG. 4A) (i.e., at a 50-fold reduction in the level of the SEA-TGT antibody to drive the same response rate).

In the Renca model, in contrast, a 1 mg/kg dose of SEA-TGT mIgG2a drove curative responses (FIG. 5C), while a similar response was seen with a 0.1 mg/kg dose when given with an anti-PD-1 antibody (FIG. 4B).

With anti-PD-1 antibody treatment alone, a dose of 5 mg/kg reduced tumor growth in the MC38 model (FIG. 5D), a dose of 10 mg/kg more moderately reduced tumor growth in the CT26 model (FIG. 5E), and a dose of 1 mg/kg did not reduce tumor growth in the Renca model (FIG. 5F). Despite very minimal single agent activity, addition of the SEA-TGT to the anti-PD-1 treatment in all of these models at substantially lower doses was able to greatly increase the anti-tumor activity.

Collectively, the results presented in the forgoing Examples support the surprising finding that cancers expressing low levels of PD-L1 can be treated by anti-TIGIT antibodies, and anti-TIGIT antibodies in combination with PD-1/PD-L1 inhibitors. As demonstrated by the foregoing examples, this was particularly found to be the case when using antibodies having enhanced Fc binding characteristics and effector function (e.g., SEA-TGT). The desired Fc binding characteristics included activities such as enhanced binding to activating FcγRs, decreased binding to inhibitory FcγRs, enhanced ADCC activity, and/or enhanced ADCP activity. Certain such antibodies with the desired activities were nonfucosylated, such as SEA-TGT. The data provided herein, for example, support the use of anti-TIGIT antibodies having an enhanced Fc backbone (e.g., SEA-TGT) in combination with anti-PD1 or anti-PD-L1 antibodies to treat patients with tumors expressing PD-L1 below the cutoff levels in currently approved therapies using anti-PD1 or anti-PD-L1 antibodies. The data further support such combination therapy in patients that are also relatively unresponsive to standard treatments with anti-PD1 or anti-PDL1 antibody treatments because of mutations in their tumors, such as those described herein.

The data provided herein also demonstrate that treatment with anti-TIGIT antibodies, for example, anti-TIGIT antibodies with enhanced effector function, such as SEA-TGT, and sub-therapeutic doses of PD-L1 inhibitors exhibit a synergistic improvement in efficacy. The data also indicate that sub-therapeutic doses of such anti-TIGIT antibodies can be used in combination with PD-1/PD-L1 inhibitors to treat cancers that express low levels of PD-L1. The ability to dose at lower levels of the anti-PD1 (or anti-PD-L1) antibody and/or the anti-TIGIT antibody can potentially lessen toxicity.

Without intending to be bound by theory, in the case of TIGIT, it is believed that nonfucosylated anti-TIGIT antibodies increase the strength of immune synapses between antigen (+) T cells and antigen presenting cells. Engagement of the FcγRIIIa on the innate cell increases their activation and production of factors that can enhance an antigen specific T cell response. The nonfucosylated backbone can, independently of the target antigen, bind to innate immune cells or other FcγRIIIa expressing cells, such as gamma delta T cells, to induce an activated state that can help elicit a secondary antigen specific T cell response. All these mechanisms by which the nonfucosylated antibody work can lead to a T cell response that drives anti-tumor activity and long-lived immune protection. The decreased or lack of binding to FcγRIIb means that there are no counter or inhibitory signals that reduce the immune activation driven by the nonfucosylated antibodies.

All publications, patents, patent applications or other documents cited herein are hereby incorporated by reference in their entirety for all purposes to the same extent as if each individual publication, patent, patent application, or other document was individually indicated to be incorporated by reference for all purposes.

VIII
Table of Sequences

| | SEQ | |
|---|---|---|
| | ID | |
| Name | NO | Sequence |
| Anti-TIGIT | 1 | QVQLVQSGAEVKKPGSSVKVSCKASGGTFSSYAISWVRQAPG |
| antibody Clone | | QGLEWMGSIIPIFGTANYAQKFQGRVTITADESTSTAYMELSS |
| 13 VH Protein | | LRSEDTAVYYCARGPSEVGAILGYVWFDPWGQGTLVTVSS |
| Anti-TIGIT | 2 | QVQLVQSGAEVKKPGSSVKVSCKASGGTFLSSAISWVRQAPG |
| antibody Clone | | QGLEWMGSLIPYFGTANYAQKFQGRVTITADESTSTAYMELS |
| 13A VH | | SLRSEDTAVYYCARGPSEVGAILGYVWFDPWGQGTLVTVSS |
| Anti-TIGIT | 3 | QVQLVQSGAEVKKPGSSVKVSCKASGGTFSAWAISWVRQAP |
| antibody Clone | | GQGLEWMGSIIPYFGKANYAQKFQGRVTITADESTSTAYMEL |
| 13B VH | | SSLRSEDTAVYYCARGPSEVSGILGYVWFDPWGQGTLVTVSS |
| Anti-TIGIT | 4 | QVQLVQSGAEVKKPGSSVKVSCKASGGTFLSSAISWVRQAPG |
| antibody Clone | | QGLEWMGSIIPLFGKANYAQKFQGRVTITADESTSTAYMELSS |
| 13C VH | | LRSEDTAVYYCARGPSEVKGILGYVWFDPWGQGTLVTVSS |
| Anti-TIGIT | 5 | QVQLVQSGAEVKKPGSSVKVSCKASGGTFLSSAISWVRQAPG |
| antibody Clone | | QGLEWMGSIIPYFGKANYAQKFQGRVTITADESTSTAYMELS |
| 13D VH | | SLRSEDTAVYYCARGPSEVKGILGYVWFDPWGQGTLVTVSS |
| Clones 13, 13A, | 6 | DIVMTQSPLSLPVTPGEPASISCRSSQSLLHSNGYNYLDWYLQ |
| 13B, 13C, and | | KPGQSPQLLIYLGSNRASGVPDRFSGSGSGTDFTLKISRVEAED |
| 13D VL Protein | | VGVYYCMQARRIPITFGGGTKVEIK |
| Clone 13 VH | 7 | GTFSSYAIS |
| CDR1 | | |
| Clones 13A, | 8 | GTFLSSAIS |
| 13C, and 13D | | |
| VH CDR1 | | |
| Clone 13B VH | 9 | GTFSAWAIS |
| CDR1 | | |
| Clone 13 VH | 10 | SIIPIFGTANYAQKFQG |
| CDR2 | | |
| Clone 13A VH | 11 | SLIPYFGTANYAQKFQG |
| CDR2 | | |
| Clones 13B and | 12 | SIIPYFGKANYAQKFQG |
| 13D VH CDR2 | | |
| Clone 13C VH | 13 | SIIPLFGKANYAQKFQG |
| CDR2 | | |
| Clones 13 and | 14 | ARGPSEVGAILGYVWFDP |
| 13A VH CDR3 | | |
| Clone 13B VH | 15 | ARGPSEVSGILGYVWFDP |
| CDR3 | | |
| Clones 13C and | 16 | ARGPSEVKGILGYVWFDP |
| 13D VH CDR3 | | |
| Clones 13, 13A, | 17 | RSSQSLLHSNGYNYLD |
| 13B, 13C, and | | |
| 13D VL CDR1 | | |
| Clones 13, 13A, | 18 | LGSNRAS |
| 13B, 13C, and | | |
| 13D VL CDR2 | | |
| Clones 13, 13A, | 19 | MQARRIPIT |
| 13B, 13C, and | | |
| 13D VL CDR3 | | |
| Clone 13 heavy | 20 | QVQLVQSGAEVKKPGSSVKVSCKASGGTFSSYAISWVRQA |
| chain hIgG1 (and | | PGQGLEWMGSIIPIFGTANYAQKFQGRVTITADESTSTAY |
| hIgG1 | | MELSSLRSEDTAVYYCARGPSEVGAILGYVWFDPWGQGT |
| nonfucosylated) | | LVTVSSASTKGPSVFPLAPSSKSTSGGTAALGCLVKDYFPEPV |
| amino acid | | TVSWNSGALTSGVHTFPAVLQSSGLYSLSSVVTVPSSSLGTQT |
| sequence; bold | | YICNVNHKPSNTKVDKKVEPKSCDKTHTCPPCPAPELLGGPSV |
| indicates VH; | | FLFPPKPKDTLMISRTPEVTCVVVDVSHEDPEVKFNWYVDGV |
| SEA-TGT | | EVHNAKTKPREEQYNSTYRVVSVLTVLHQDWLNGKEYKCK |
| | | VSNKALPAPIEKTISKAKGQPREPQVYTLPPSRDELTKNQVSLT |
| | | CLVKGFYPSDIAVEWESNGQPENNYKTTPPVLDSDGSFFLYSK |
| | | LTVDKSRWQQGNVFSCSVMHEALHNHYTQKSLSLSPGK |
| Clone 13A heavy | 21 | QVQLVQSGAEVKKPGSSVKVSCKASGGTFLSSAISWVRQA |
| chain hIgG1 (and | | PGQGLEWMGSLIPYFGTANYAQKFQGRVTITADESTSTAY |
| hIgG1 | | MELSSLRSEDTAVYYCARGPSEVGAILGYVWFDPWGQGT |
| nonfucosylated) | | LVTVSSASTKGPSVFPLAPSSKSTSGGTAALGCLVKDYFPEPV |
| amino acid | | TVSWNSGALTSGVHTFPAVLQSSGLYSLSSVVTVPSSSLGTQT |
| sequence; bold | | YICNVNHKPSNTKVDKKVEPKSCDKTHTCPPCPAPELLGGPSV |
| indicates VH | | FLFPPKPKDTLMISRTPEVTCVVVDVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSRDELTKNQVSLT |
| | | CLVKGFYPSDIAVEWESNGQPENNYKTTPPVLDSDGSFFLYSK |
| | | LTVDKSRWQQGNVFSCSVMHEALHNHYTQKSLSLSPGK |
| Clone 13B heavy | 22 | QVQLVQSGAEVKKPGSSVKVSCKASGGTFSAWAISWVRQ |
| chain hIgG1 (and | | APGQGLEWMGSIIPYFGKANYAQKFQGRVTITADESTSTA |
| hIgG1 | | YMELSSLRSEDTAVYYCARGPSEVSGILGYVWFDPWGQG |
| nonfucosylated) | | TLVTVSSASTKGPSVFPLAPSSKSTSGGTAALGCLVKDYFPEP |
| amino acid | | VTVSWNSGALTSGVHTFPAVLQSSGLYSLSSVVTVPSSSLGTQ |
| sequence; bold | | TYICNVNHKPSNTKVDKKVEPKSCDKTHTCPPCPAPELLGGPS |
| indicates VH | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGK |
| Clone 13C heavy | 23 | QVQLVQSGAEVKKPGSSVKVSCKASGGTFLSSAISWVRQA |
| chain hIgG1 (and | | PGQGLEWMGSIIPLFGKANYAQKFQGRVTITADESTSTAY |
| hIgG1 | | MELSSLRSEDTAVYYCARGPSEVKGILGYVWFDPWGQGT |
| nonfucosylated) | | LVTVSSASTKGPSVFPLAPSSKSTSGGTAALGCLVKDYFPEPV |
| amino acid | | TVSWNSGALTSGVHTFPAVLQSSGLYSLSSVVTVPSSSLGTQT |
| sequence; bold | | YICNVNHKPSNTKVDKKVEPKSCDKTHTCPPCPAPELLGGPSV |
| indicates VH | | FLFPPKPKDTLMISRTPEVTCVVVDVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSRDELTKNQVSLT |
| | | CLVKGFYPSDIAVEWESNGQPENNYKTTPPVLDSDGSFFLYSK |
| | | LTVDKSRWQQGNVFSCSVMHEALHNHYTQKSLSLSPGK |
| Clone 13D heavy | 24 | QVQLVQSGAEVKKPGSSVKVSCKASGGTFLSSAISWVRQA |
| chain hIgG1 (and | | PGQGLEWMGSIIPYFGKANYAQKFQGRVTITADESTSTAY |
| hIgG1 | | MELSSLRSEDTAVYYCARGPSEVKGILGYVWFDPWGQGT |
| nonfucosylated) | | LVTVSSASTKGPSVFPLAPSSKSTSGGTAALGCLVKDYFPEPV |
| amino acid | | TVSWNSGALTSGVHTFPAVLQSSGLYSLSSVVTVPSSSLGTQT |
| sequence; bold | | YICNVNHKPSNTKVDKKVEPKSCDKTHTCPPCPAPELLGGPSV |
| indicates VH | | FLFPPKPKDTLMISRTPEVTCVVVDVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSRDELTKNQVSLT |
| | | CLVKGFYPSDIAVEWESNGQPENNYKTTPPVLDSDGSFFLYSK |
| | | LTVDKSRWQQGNVFSCSVMHEALHNHYTQKSLSLSPGK |
| Clone 13, 13A, | 25 | DIVMTQSPLSLPVTPGEPASISCRSSQSLLHSNGYNYLDWY |
| 13B, 13C, and | | LQKPGQSPQLLIYLGSNRASGVPDRFSGSGSGTDFTLKISR |
| 13D light chain | | VEAEDVGVYYCMQARRIPITFGGGTKVEIKRTVAAPSVFIF |
| hkappa (and | | PPSDEQLKSGTASVVCLLNNFYPREAKVQWKVDNALQSGNS |
| nonfucosylated) | | QESVTEQDSKDSTYSLSSTLTLSKADYEKHKVYACEVTHQGL |
| amino acid | | SSPVTKSFNRGEC |
| sequence; bold | | |
| indicates VL; | | |
| SEA-TGT | | |

What is claimed is:

1. A method of treating cancer, comprising administering to a subject with cancer (1) an anti-TIGIT antibody, and (2) an anti-PD-1 antibody or an anti-PD-L1 antibody; wherein the level of PD-L1 in a sample of the cancer is less than 10 as measured by Combined Positive Score (CPS), or less than 50% as measured by Total Proportion Score (TPS), or less than 50% as measured by a Tumor Cell score (TC), or less than 10% as measured by Tumor-Infiltrating Immune Cell staining (IC), and wherein the anti-TIGIT antibody comprises an Fc region with enhanced effector function.

2. The method of claim 1, wherein the cancer expresses a level of PD-L1 that is less than 5, or less than 3, or less than 1, as measured by CPS.

3. The method of claim 1 or claim 2, wherein the cancer expresses a level of PD-L1 that is less than 40%, or less than 30%, or less than 20%, or less than 10%, or less than 5%, or less than 3%, or less than 1%, as measured by TPS.

4. The method of any one of claims 1-3, wherein the cancer expresses a level of PD-L1 that is less than 40%, or less than 30%, or less than 20%, or less than 10%, or less than 5%, or less than 3%, or less than 1%, as measured by TC.

5. The method of any one of claims 1-4, wherein the cancer expresses a level of PD-L1 that is less than 5%, or less than 3%, or less than 1%, as measured by IC.

6. The method of any one of claims 1-5, wherein:
a) the cancer is non-small lung cancer, and the TPS is <1%;
b) the cancer is head and neck squamous cell cancer (HNSCC) and the CPS is <1;
c) the cancer is urothelial carcinoma and the CPS is <10;
d) the cancer is gastric cancer and the CPS is <1;
e) the cancer is esophageal cancer and the CPS<10;
f) the cancer is cervical cancer and the CPS<1; or
g) the cancer is triple negative breast cancer, and the CPS<10.

7. The method of claim 6, wherein the method comprises administering an anti-PD-1 antibody, wherein the anti-PD-1 antibody is pembrolizumab or nivolumab.

8. The method of any one of claims 1-5, wherein the cancer is non-small cell lung cancer, and the TPS is <50%.

9. The method of claim 8, the method comprises administering an anti-PD-1 antibody, wherein the anti-PD-1 antibody is cemiplimab.

10. The method of any one of claims 1-5, wherein:
a) the cancer is urothelial carcinoma and IC is <5%;
b) the cancer is triple-negative breast cancer and IC is <1%; or
c) the cancer is non-small cell lung cancer and IC is <10%; or
d) the cancer is non-small cell lung cancer and TC<50%.

11. The method of claim 10, the method comprises administering an anti-PD-1 antibody, wherein the anti-PD-1 antibody is atezolizumab.

12. The method of any one of claims 1-11, wherein the anti-PD-1 antibody or anti-PD-L1 antibody is administered at a sub-therapeutic dose.

13. A method of treating cancer, comprising administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the anti-PD-1 antibody or anti-PD-L1 antibody is administered at a sub-therapeutic dose.

14. The method of claim 12 or claim 13, wherein the sub-therapeutic dose of the anti-PD-1 antibody or anti-PD-L1 antibody: a) is lower than the monotherapy dose of the antibody for the cancer being treated and/or b) comprises less frequent dosing of the antibody than the frequency of monotherapy dosing for the cancer being treated.

15. The method of any one of claims 12-14, wherein the sub-therapeutic dose of the antibody includes a dose that is lower than the monotherapy dose of the antibody for the cancer being treated.

16. The method of claim 15, wherein the sub-therapeutic dose is a dose of the antibody that is between 5% and 90%, or 5% and 80%, or 5% and 70%, or 5% and 60%, or 5% and 50%, or 5% and 40%, or 5% and 30% of the monotherapy dose for the cancer being treated.

17. The method of any one of claims 14-16, wherein the method comprises administering an anti-PD-1 antibody, wherein the anti-PD-1 antibody is pembrolizumab, and wherein the monotherapy dose is 200 mg or 400 mg.

18. The method of any one of claims 14-16, wherein the method comprises administering an anti-PD-1 antibody, wherein the anti-PD-1 antibody is nivolumab, and wherein the monotherapy dose is 240 mg, 360 mg, or 480 mg.

19. The method of any one of claims 14-16, wherein the method comprises administering an anti-PD-1 antibody, wherein the anti-PD-1 antibody is cemiplimab, and wherein the monotherapy dose is 350 mg.

20. The method of any one of claims 14-16, wherein the method comprises administering an anti-PD-L1 antibody, wherein the anti-PD-L1 antibody is avelumab, and wherein the monotherapy dose is 800 mg.

21. The method of any one of claims 14-16, wherein the method comprises administering an anti-PD-L1 antibody, wherein the anti-PD-L1 antibody is durvalumab, and wherein the monotherapy dose is 10 mg/kg or 1500 mg.

22. The method of any one of claims 14-16, wherein the method comprises administering an anti-PD-L1 antibody, wherein the anti-PD-L1 antibody is atezolizumab, and wherein the monotherapy dose is 840 mg, 1200 mg, or 1680 mg.

23. The method of any one of claims 12-22, wherein the sub-therapeutic dose of the antibody comprises less frequent dosing of the antibody than the frequency of monotherapy dosing for the cancer being treated.

24. The method of claim 23, wherein the method comprises administering an anti-PD-1 antibody, wherein the anti-PD-1 antibody is pembrolizumab, and wherein the frequency of monotherapy dosing is every 3 weeks or every 6 weeks.

25. The method of claim 24, wherein the method comprises administering an anti-PD-1 antibody, wherein the anti-PD-1 antibody is pembrolizumab, and wherein the monotherapy dose is 200 mg every 3 weeks or 400 mg every 6 weeks.

26. The method of claim 23, wherein the method comprises administering an anti-PD-1 antibody, wherein the anti-PD-1 antibody is nivolumab, and wherein the frequency of monotherapy dosing is every 2 weeks or every 3 weeks or every 4 weeks.

27. The method of claim 26, wherein the method comprises administering an anti-PD-1 antibody, wherein the anti-PD-1 antibody is nivolumab, and wherein the monotherapy dose is 240 mg every 2 weeks, 360 mg every 3 weeks, or 480 mg every 4 weeks.

28. The method of claim 23, wherein the method comprises administering an anti-PD-1 antibody, wherein the anti-PD-1 antibody is cemiplimab, and wherein the frequency of monotherapy dosing is every 3 weeks.

29. The method of claim 23, wherein the method comprises administering an anti-PD-L1 antibody, wherein the anti-PD-L1 antibody is avelumab, wherein the frequency of monotherapy dosing is every 2 weeks.

30. The method of claim 23, wherein the method comprises administering an anti-PD-L1 antibody, wherein the anti-PD-L1 antibody is durvalumab, wherein the frequency of monotherapy dosing is every 2 weeks or every 4 weeks.

31. The method of claim 30, wherein the method comprises administering an anti-PD-L1 antibody, wherein the anti-PD-L1 antibody is durvalumab, and wherein the monotherapy dose is 10 mg/kg mg every 2 weeks or 1500 mg every 4 weeks.

32. The method of claim 23, wherein the method comprises administering an anti-PD-L1 antibody, wherein the anti-PD-L1 antibody is atezolizumab, wherein the frequency of monotherapy dosing is every 2 weeks, every 3 weeks, or every 4 weeks.

33. The method of claim 32, wherein the method comprises administering an anti-PD-L1 antibody, wherein the anti-PD-L1 antibody is atezolizumab, and wherein the monotherapy dose is 840 mg every 2 weeks, 1200 mg every 3 weeks, or 1680 mg every 4 weeks.

34. The method of any one of claims 1-33, wherein the cancer is selected from small cell lung cancer, early-stage small cell lung cancer, renal cell carcinoma, urothelial cancer, triple negative breast cancer, gastric cancer, hepatocellular carcinoma, glioblastoma, ovarian cancer, head and neck squamous cell carcinoma, esophageal squamous cell carcinoma (ESCC), and non-microsatellite instability high (non-MSI high) colorectal cancer.

35. A method of treating cancer, comprising administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer is selected from small cell lung cancer, early-stage small cell lung cancer, renal cell carcinoma, urothelial cancer, triple negative breast cancer, gastric cancer, hepatocellular carcinoma, glioblastoma, ovarian cancer, head and neck squamous cell carcinoma, esophageal squamous cell carcinoma (ESCC), and non-microsatellite instability high (non-MSI high) colorectal cancer.

36. The method of claim 34 or claim 35, wherein the method is first line treatment of urothelial cancer.

37. The method of any one of claims 1-36, wherein the cancer comprises a mutation that reduces the efficacy of the anti-PD-1 antibody or anti-PD-L1 antibody.

38. A method of treating cancer, comprising administering to a subject with cancer (1) an anti-TIGIT antibody, and (2) an anti-PD-1 antibody or an anti-PD-L1 antibody; wherein the anti-TIGIT antibody comprises an Fc region with enhanced effector function, and wherein the cancer comprises a mutation that reduces the efficacy of the anti-PD-1 antibody or anti-PD-L1 antibody.

39. The method of claim 37 or claim 38, wherein the cancer comprises a mutation in an EGFR gene and/or a mutation in an ALK gene and/or a mutation in the ROS1 gene.

40. The method of any one of claims 37-39, wherein the cancer is non-small cell lung cancer, and wherein the cancer comprises a mutation in an EGFR gene and/or a mutation in an ALK gene.

41. The method of claim 40, wherein the method comprises administering an anti-PD-1 antibody, wherein the anti-PD-1 antibody is pembrolizumab or nivolumab; or wherein the method comprises administering an anti-PD-L1 antibody, wherein the anti-PD-L1 antibody is atezolizumab.

42. The method of any one of the preceding claims, wherein the anti-TIGIT antibody comprises an Fc with enhanced binding to at least one of FcγRIIIa, FcγRIIa, and FcγRI.

43. The method of claim 42, wherein the anti-TIGIT antibody comprises an Fc with enhanced binding to at least FcγRIIIa.

44. The method of claim 42, wherein anti-TIGIT antibody comprises an Fc with enhanced binding to at least FcγRIIIa and FcγRIIa.

45. The method of claim 42, wherein the anti-TIGIT antibody comprises an Fc with enhanced binding to at least FcγRIIIa and FcγRI.

46. The method of claim 42, wherein the anti-TIGIT antibody comprises an Fc with enhanced binding to FcγRIIIa, FcγRIIa, and FcγRI.

47. The method of any one of claims 42-46, wherein the Fc of the anti-TIGIT antibody has reduced binding to FcγRIIb.

48. The method of any one of the preceding claims, wherein the anti-TIGIT antibody comprises substitutions S293D, A330L, and I332E in the heavy chain constant region.

49. The method of any one of the preceding claims, wherein the anti-TIGIT antibody is nonfucosylated.

50. The method of any one of the preceding claims, wherein the method comprises administering a composition of anti-TIGIT antibodies, wherein at least 90%, at least 91%, at least 92%, at least 93%, at least 94%, at least 95%, at least 96%, at least 97%, at least 98%, or at least 99% of the antibodies in the composition are nonfucosylated.

51. The method of any one of the preceding claims, wherein the Fc of the anti-TIGIT antibody comprises an Fc with enhanced ADCC and/or ADCP activity relative to a corresponding wild-type Fc of the same isotype.

52. The method of any one of the preceding claims, wherein the anti-TIGIT antibody comprises:
a) a heavy chain CDR1 comprising an amino acid sequence selected from SEQ ID NOs: 7-9;
b) a heavy chain CDR2 comprising an amino acid sequence selected from SEQ ID NOs: 10-13;
c) a heavy chain CDR3 comprising an amino acid sequence selected from SEQ ID NOs: 14-16;
d) a light chain CDR1 comprising the amino acid sequence of SEQ ID NO: 17;
e) a light chain CDR2 comprising the amino acid sequence of SEQ ID NO: 18; and
f) a light chain CDR3 comprising the amino acid sequence of SEQ ID NO: 19.

53. The method of any one of the preceding claims, wherein the anti-TIGIT antibody comprises a heavy chain CDR1, CDR2, and CDR3 and a light chain CDR1, CDR, and CDR3 comprising the sequences of:
a) SEQ ID NOs: 7, 10, 14, 17, 18, and 19, respectively; or
b) SEQ ID NOs: 8, 11, 14, 17, 18, and 19, respectively; or
c) SEQ ID NOs: 9, 12, 15, 17, 18, and 19, respectively; or
d) SEQ ID NOs: 8, 13, 16, 17, 18, and 19, respectively; or
e) SEQ ID NOs: 8, 12, 16, 17, 18, and 19, respectively.

54. The method of any one of the preceding claims, wherein the anti-TIGIT antibody comprises a heavy chain variable region comprising an amino acid sequence selected from SEQ ID NOs: 1-5 and a light chain variable region comprising the amino acid sequence of SEQ ID NO: 6.

55. The method of any one of the preceding claims, wherein the anti-TIGIT antibody comprises a heavy chain comprising an amino acid sequence selected from SEQ ID NOs: 20-24 and a light chain comprising the amino acid sequence of SEQ ID NO: 25.

56. The method of any one of the preceding claims, wherein the anti-TIGIT antibody is administered at a sub-therapeutic dose.

57. The method of claim 56, wherein the sub-therapeutic dose of the anti-TIGIT antibody a) is lower than the monotherapy dose of the anti-TIGIT antibody for the cancer being treated and/or b) comprises less frequent dosing of the anti-TIGIT antibody than the frequency of monotherapy dosing for the cancer being treated.

58. The method of claim 56 or claim 57, wherein the sub-therapeutic dose of the anti-TIGIT antibody includes a dose that is lower than the monotherapy dose of the anti-TIGIT antibody for the cancer being treated.

59. The method of any one of claims 56-58, wherein the sub-therapeutic dose is a dose of the anti-TIGIT antibody that is between 5% and 90%, or 5% and 80%, or 5% and 70%, or 5% and 60%, or 5% and 50%, or 5% and 40%, or 5% and 30% of the monotherapy dose for the cancer being treated.

60. The method of any one of claims 56-59, wherein the sub-therapeutic dose of the anti-TIGIT antibody comprises less frequent dosing of the anti-TIGIT antibody than the frequency of monotherapy dosing for the cancer being treated.

61. The method of any one of the preceding claims, wherein the method comprises administering an anti-PD-1 antibody.

62. The method of claim 61, wherein the anti-PD-1 antibody is selected from pembrolizumab, nivolumab, CT-011, BGB-A317, cemiplimab, sintilimab, tislelizumab, TSR-042, PDR001, or toripalimab.

63. The method of any one of claims 1-60, wherein the method comprises administering an anti-PD-L1 antibody.

64. The method of claim 63, wherein the anti-PD-L1 antibody is selected from durvalumab, BMS-936559, atezolizumab, or avelumab.


SEQUENCE LISTING (25 sequences)

SEQ ID NO: 1 - protein, 125 aa, synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 2 - protein, 125 aa, synthetic construct
QVQLVQSGAEVKKPGSSVKVSCKASGGTFLSSAISWVRQAPGQGLEWMGSLIPYFGTANYAQKFQGRVTITADESTSTAYMELSSLRSEDTAVYYCARGPSEVGAILGYVWFDPWGQGTLVTVSS

SEQ ID NO: 3 - protein, 125 aa, synthetic construct
QVQLVQSGAEVKKPGSSVKVSCKASGGTFSAWAISWVRQAPGQGLEWMGSIIPYFGKANYAQKFQGRVTITADESTSTAYMELSSLRSEDTAVYYCARGPSEVSGILGYVWFDPWGQGTLVTVSS

SEQ ID NO: 4 - protein, 125 aa, synthetic construct
QVQLVQSGAEVKKPGSSVKVSCKASGGTFLSSAISWVRQAPGQGLEWMGSIIPLFGKANYAQKFQGRVTITADESTSTAYMELSSLRSEDTAVYYCARGPSEVKGILGYVWFDPWGQGTLVTVSS

SEQ ID NO: 5 - protein, 125 aa, synthetic construct
QVQLVQSGAEVKKPGSSVKVSCKASGGTFLSSAISWVRQAPGQGLEWMGSIIPYFGKANYAQKFQGRVTITADESTSTAYMELSSLRSEDTAVYYCARGPSEVKGILGYVWFDPWGQGTLVTVSS

SEQ ID NO: 6 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 7 - protein, 9 aa, synthetic construct
GTFSSYAIS

SEQ ID NO: 8 - protein, 9 aa, synthetic construct
GTFLSSAIS

SEQ ID NO: 9 - protein, 9 aa, synthetic construct
GTFSAWAIS

SEQ ID NO: 10 - protein, 17 aa, synthetic construct
SIIPIFGTANYAQKFQG

SEQ ID NO: 11 - protein, 17 aa, synthetic construct
SLIPYFGTANYAQKFQG

SEQ ID NO: 12 - protein, 17 aa, synthetic construct
SIIPYFGKANYAQKFQG

SEQ ID NO: 13 - protein, 17 aa, synthetic construct
SIIPLFGKANYAQKFQG

SEQ ID NO: 14 - protein, 18 aa, synthetic construct
ARGPSEVGAILGYVWFDP

SEQ ID NO: 15 - protein, 18 aa, synthetic construct
ARGPSEVSGILGYVWFDP

SEQ ID NO: 16 - protein, 18 aa, synthetic construct
ARGPSEVKGILGYVWFDP

SEQ ID NO: 17 - protein, 16 aa, synthetic construct
RSSQSLLHSNGYNYLD

SEQ ID NO: 18 - protein, 7 aa, synthetic construct
LGSNRAS

SEQ ID NO: 19 - protein, 9 aa, synthetic construct
MQARRIPIT

SEQ ID NO: 20 - protein, 455 aa, synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 455 aa, synthetic construct
QVQLVQSGAEVKKPGSSVKVSCKASGGTFLSSAISWVRQAPGQGLEWMGSLIPYFGTANYAQKFQGRVTITADESTSTAYMELSSLRSEDTAVYYCARGPSEVGAILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 455 aa, synthetic construct
QVQLVQSGAEVKKPGSSVKVSCKASGGTFSAWAISWVRQAPGQGLEWMGSIIPYFGKANYAQKFQGRVTITADESTSTAYMELSSLRSEDTAVYYCARGPSEVSGILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 455 aa, synthetic construct
QVQLVQSGAEVKKPGSSVKVSCKASGGTFLSSAISWVRQAPGQGLEWMGSIIPLFGKANYAQKFQGRVTITADESTSTAYMELSSLRSEDTAVYYCARGPSEVKGILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 455 aa, synthetic construct
QVQLVQSGAEVKKPGSSVKVSCKASGGTFLSSAISWVRQAPGQGLEWMGSIIPYFGKANYAQKFQGRVTITADESTSTAYMELSSLRSEDTAVYYCARGPSEVKGILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC